(19) United States
(12) Patent Application Publication
(10) Pub. No.: US 2009/0093463 A1
(43) Pub. Date: Apr. 9, 2009

(54) NOVEL COMPOUNDS OF SUBSTITUTED AND UNSUBTITUTED ADAMANTYL AMIDES

(75) Inventors: Hengmiao Cheng, San Diego, CA (US); Bridget McCarthy Cole, Quincy, MA (US)

Correspondence Address: PFIZER INC, 10555 SCIENCE CENTER DRIVE, SAN DIEGO, CA, 92121, US

(73) Assignee: AGOURON PHARMACEUTICALS INC., San Diego, CA (US)

(21) Appl. No.: 11/718,327

(22) PCT Filed: Oct. 20, 2005
(86) PCT No.: PCT/IB2005/003311

§ 371 Date: Apr. 30, 2007

Related U.S. Application Data
(60) Provisional application No. 60/624,591, filed on Nov. 2, 2004.

(51) Int. Cl. A61K 31/551; C07D 211/46; C07D 243/08; A61K 31/164; C07C 233/37; A61K 31/445

(52) U.S. Cl. 514/218; 546/221; 514/327; 540/575; 564/191; 514/613

(57) ABSTRACT

The present invention relates to compounds with the formula (I) or a pharmaceutically acceptable salt thereof: The invention also relates to pharmaceutical compositions comprising the compounds of formula (I) and methods of treating a condition that is mediated by the modulation of 11-β-hsd-1, the method comprising administering to a mammal an effective amount of a compound of formula (I).

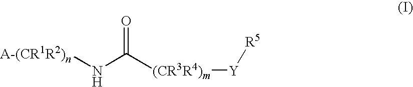

### FIELD OF THE INVENTION

The present invention relates to novel compounds, to pharmaceutical compositions comprising the compounds, as well as to the use of the compounds in medicine and for the preparation of a medicament which acts on the human 11-β-hydroxysteroid dehydrogenase type 1 enzyme (11-β-hsd-1).

### BACKGROUND OF THE INVENTION

It has been known for more than half a century that glucocorticoids have a central role in diabetes. For example, the removal of the pituitary or the adrenal gland from a diabetic animal alleviates the most severe symptoms of diabetes and lowers the concentration of glucose in the blood (Long, C. D. and F. D. W. Leukins (1936) J. Exp. Med. 63: 465-490; Houssay, B. A. (1942) Endocrinology 30: 884-892). Additionally, it is also well established that glucocorticoids enable the effect of glucagon on the liver.

The role of 11-β-hsd-1 as an important regulator of local glucocorticoid effects and thus of hepatic glucose production is well substantiated (see e.g. Jamieson et al. (2000) J. Endocrinol. 165: p. 685-692). The hepatic insulin sensitivity was improved in healthy human volunteers treated with the non-specific 11-β-hsd-1 inhibitor carbenoxolone (Walker, B. R., et al. (1995) J. Clin. Endocrinol. Metab. 80: 3155-3159). Furthermore, the expected mechanism has been established by different experiments with mice and rats. These studies showed that the mRNA levels and activities of two key enzymes in hepatic glucose production were reduced, namely the rate-limiting enzyme in gluconeogenesis, phosphoenolpyruvate carboxykinase (PEPCK), and glucose-6-phosphatase (G6Pase) catalyzing the last common step of gluconeogenesis and glycogenolysis. Finally, the blood glucose level and hepatic glucose production was reduced in mice having the 11-β-hsd-1 gene knocked-out. Data from this model also confirms that inhibition of 11-β-hsd-1 will not cause hypoglycemia, as predicted, since the basal levels of PEPCK and G6Pase are regulated independently of glucocorticoids (Kotelevtsev, Y., et al., (1997) Proc. Natl. Acad. Sci. USA 94: 14924-14929).

Abdominal obesity is closely associated with glucose intolerance, hyperinsulinemia, hypertriglyceridemia, and other factors of the so-called Metabolic Syndrome (e.g. raised blood pressure, decreased levels of HDL and increased levels of VLDL) (Montague & O'Rahilly, Diabetes 49: 883-888, 2000). Obesity is an important factor in Metabolic Syndrome as well as in the majority (>80%) of type 2 diabetic, and omental fat appears to be of central importance. Inhibition of the enzyme in pre-adipocytes (stromal cells) has been shown to decrease the rate of differentiation into adipocytes. This is predicted to result in diminished expansion (possibly reduction) of the omental fat depot, i.e. reduced central obesity (Bujalska, I. J., Kumar, S., and Stewart, P. M. (1997) Lancet 349: 1210-1213).

The compounds of the present invention are 11β-hsd-1 inhibitors, and are therefore believed to be useful in the treatment of diabetes, obesity, glaucoma, osteoporosis, cognitive disorders, immune disorders, depression, hypertension, and metabolic diseases.

### SUMMARY OF THE INVENTION

The present invention relates to a compound of formula (I):

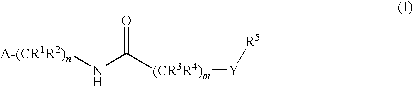

wherein:

each R¹, R², R³, and R⁴ is independently selected from H and (C₁-C₆)alkyl;

Y is selected from the group consisting of O, S, and NR⁶;

each R⁵ and R⁶ is independently selected from the group consisting of H, (C₁-C₆) alkyl, (C₂-C₆) alkenyl, (C₂-C₆) alkynyl, —(CR⁷R⁸)ₜ(C₃-C₁₀)cycloalkyl, —(CR⁷R⁸)ₜ(C₆-C₁₀)aryl, and —(CR⁷R⁸)ₜ(4-11)-membered heterocyclyl;

or, where Y is NR⁶, R⁵ and R⁶ may optionally be taken together with the nitrogen atom to which they are attached to form a (4-11)-membered heterocyclyl, and the (4-11)-membered heterocyclyl may optionally be substituted by 1 to 5 R⁹ groups;

each R⁷ and R⁸ is independently selected from the group consisting of H, (C₁-C₆) alkyl, (C₂-C₆) alkenyl, and (C₂-C₆) alkynyl;

A is adamantyl;

n and m are independently selected from the group consisting of 0, 1, 2, and 3;

k is 1 or 2;

j is selected from the group consisting of 0, 1, and 2;

t, u, p, q and v are each independently selected from the group consisting of 0, 1, 2, 3, 4, and 5;

any carbon atom of A, R¹, R², R³, R⁴, R⁵, R⁶, R⁷, and, R⁸ may optionally substituted by 1 to 5 R⁹ groups;

any nitrogen atom of R⁵ or R⁶ wherein R⁵ or R⁶ is a (4-11)-membered heterocyclyl are each optionally substituted by 1 to 5 R⁹ groups;

each R⁹ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR¹⁰R¹¹)ₜ(C₃-C₁₀)cycloalkyl, —(CR¹²R¹³)ₜ(C₆-C₁₀)aryl, —O—R¹², —(C═O)—R¹², —(C═O)—O—R¹², —O—(C═O)—R¹², —O—(R¹²)—O—(R¹³), —NR¹²(C═O)—R¹³, —(C═O)—NR¹²R¹³, —NR¹²R¹³, —NR¹²OR¹³, —S(O)ₖNR¹²R¹³, —S(O)ⱼ(C₁-C₆)alkyl, —O—SO₂—R¹⁴, —NR¹⁴—S(O)ₖ—R¹⁵, —(CR¹⁴R¹⁵)ᵥ(C₆-C₁₀) aryl, —(CR¹⁴R¹⁵)ᵥ(4-11)-membered heterocyclyl, —(CR¹⁴R¹⁵)_{q}(C═O)(CR¹⁴R¹⁵)ᵥ(C₆-C₁₀)aryl, —(CR¹⁴R¹⁵)_{q}(C═O)(CR¹⁴R¹⁵)ᵥ(4-11)-membered heterocyclyl, —(CR¹⁴R¹⁵)ᵥO(CR¹⁴R¹⁵)_{q}(C₆-C₁₀)aryl, —(CR¹⁴R¹⁵)ᵥO(CR¹⁴R¹⁵)_{q}(4-11)-membered heterocyclyl, —(CR¹⁴R¹⁵)_{q}S(O)ⱼ(CR¹⁴R¹⁵)ᵥ(C₆-C₁₀)aryl, and —(CR¹⁴R¹⁵)_{q}S(O)ⱼ(CR¹⁴R¹⁵)ᵥ(4-11)-membered heterocyclyl;

any 1 or 2 carbon atoms of any (4-11)-membered heterocyclyl of the foregoing R⁹ groups are optionally substituted with an oxo (═O);

any carbon atom of any (C₁-C₆)alkyl, any (C₆-C₁₀)aryl and any (4-11)-membered heterocyclyl of the foregoing R⁹ groups may be optionally substituted with 1 to 5 substituents independently selected from halo, cyano, nitro, —CF₃, —CFH₂, —CF₂H, trifluoromethoxy, azido, —OR¹⁶, —(C═O)—R¹⁶, —(C═O)—O—R¹⁶, —O—(C═O)—R¹⁶, —NR¹⁶(C═O)—R¹⁷, —(C═O)—NR¹⁶R¹⁷, —NR¹⁶R¹⁷, —NR¹⁶OR¹⁷, —S(O)ₖNR¹²R¹³, —S(O)ⱼ(C₁-C₆)alkyl, —O—SO₂—R¹⁴, —NR¹⁴—S(O)ₖ—R¹⁵, (C₁-C₆)alkyl, (C₂-C₆)alkenyl, (C₂-C₆)alkynyl, —(CR¹⁷R¹⁸)ᵤ(C₆-C₁₀)aryl, and —(CR¹⁷R¹⁸)ᵤ(4-11)-membered heterocyclyl;

each R¹⁰, R¹¹, R¹², R¹³, R¹⁴, R¹⁵, R¹⁶, R¹⁷, and R¹⁸ group is independently selected from the group consisting of H, (C₁-C₆)alkyl, —(CR¹⁹R²⁰)ₜ(C₃-C₁₀)cycloalkyl, —(CR¹⁹R²⁰)ₚ(C₆-C₁₀)aryl, and —(CR¹⁹R²⁰)ₚ(4-11)-membered heterocyclyl;

any 1 or 2 carbon atoms of the (4-11)-membered heterocyclyl of said each R¹⁰, R¹¹, R¹², R¹³, R¹⁴, R¹⁵, R¹⁶, R¹⁷, and R¹⁸ group is optionally substituted with an oxo (═O);

any carbon atom of the (C₁-C₆)alkyl, any (C₆-C₁₀)aryl and any (4-11)-membered heterocyclyl of the foregoing R¹⁰, R¹¹, R¹², R¹³, R¹⁴, R¹⁵, R¹⁶, R¹⁷, and R¹⁸ groups are optionally substituted with 1 to 5 substituents independently selected from the group consisting of halo, cyano, nitro, —NR²¹R²², —CF₃, —CHF₂, —CH₂F, trifluoromethoxy, (C₁-C₆)alkyl, (C₂-C₆)alkenyl, (C₂-C₆)alkynyl, hydroxy, and (C₁-C₆) alkoxy;

each R¹⁹, R²⁰, R²¹, and R²² group is independently selected from the group consisting of H and (C₁-C₆)alkyl;

and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, amino, —NH(C₁-C₆)(alkyl) and —N(C₁-C₆) (alkyl)(C₁-C₆) alkyl;

or a pharmaceutically acceptable salt or solvate thereof.

An embodiment of the invention according to a compound formula (I), wherein Y is O.

Another embodiment of the invention according to a compound formula (I), wherein Y is NR⁶.

Yet another embodiment of the invention according to a compound formula (I), wherein R⁵ and R⁶ are taken together with the nitrogen to which they are attached to form a (4-11)-membered heterocyclyl.

In a particular aspect of this embodiment, wherein R⁵ and R⁶ are taken together with the nitrogen to which they are attached to form a (4-11)-membered heterocyclyl, the (4-11)-membered heterocyclyl is selected from the group consisting of

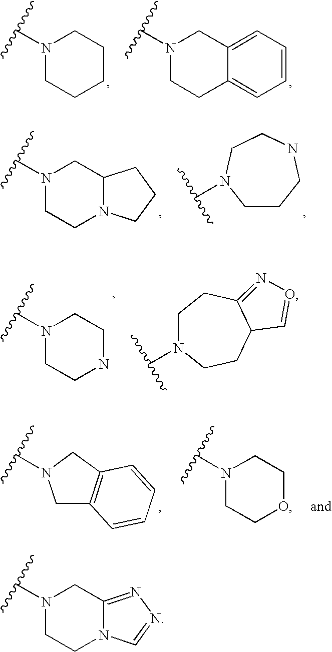

In yet another embodiment of the compound according to formula (I), wherein R⁵ and R⁶ are taken together with the nitrogen to which they are attached to form a (4-11)-membered heterocyclyl, the (4-11)-membered heterocyclyl is selected from the group consisting of pyrrolidinyl, indolyl, isoquinolinyl, piperazinyl, and piperidinyl.

An embodiment of the invention according to compound of formula (I) is selected from the group consisting of:

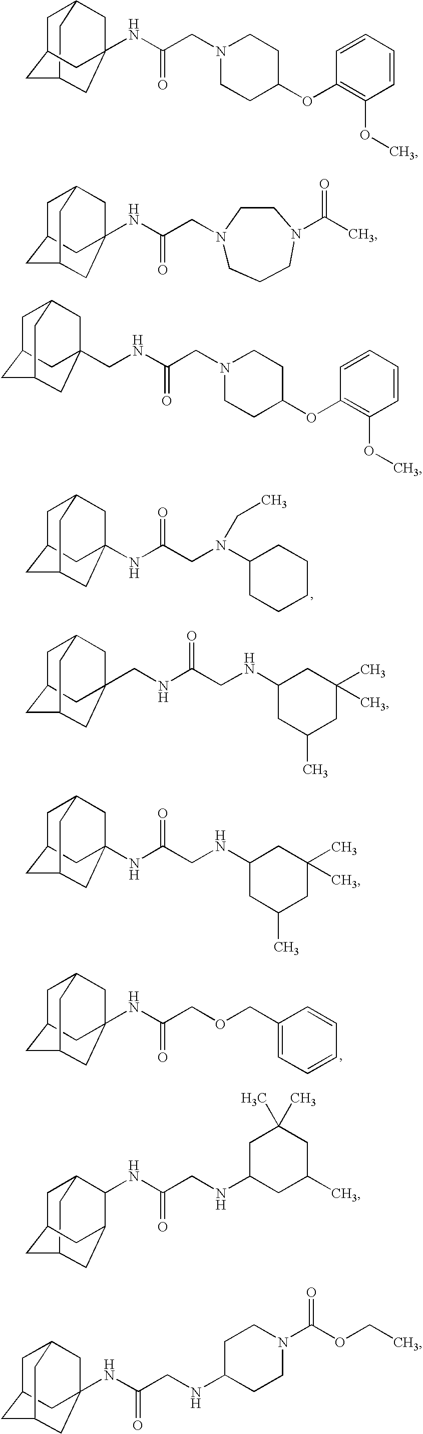

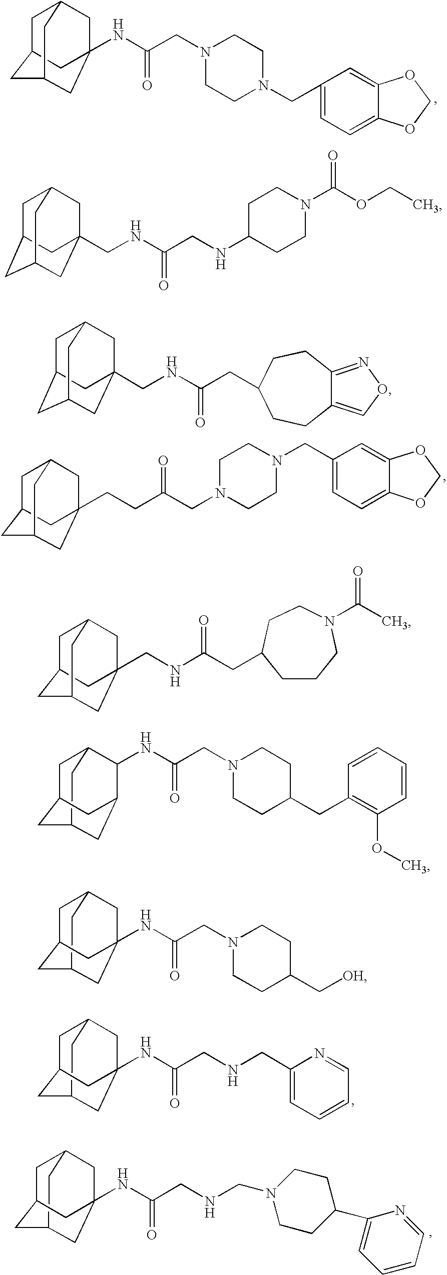

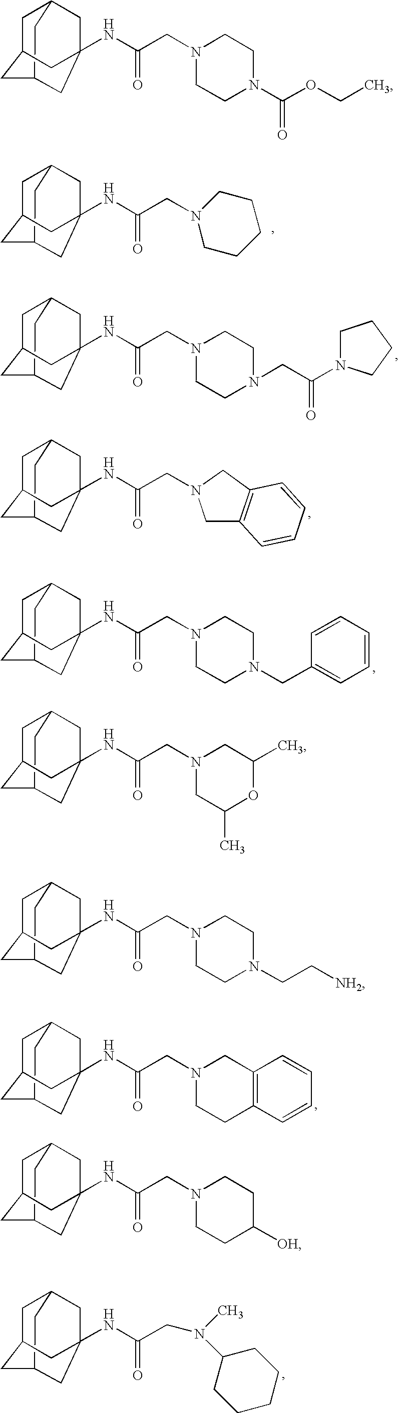

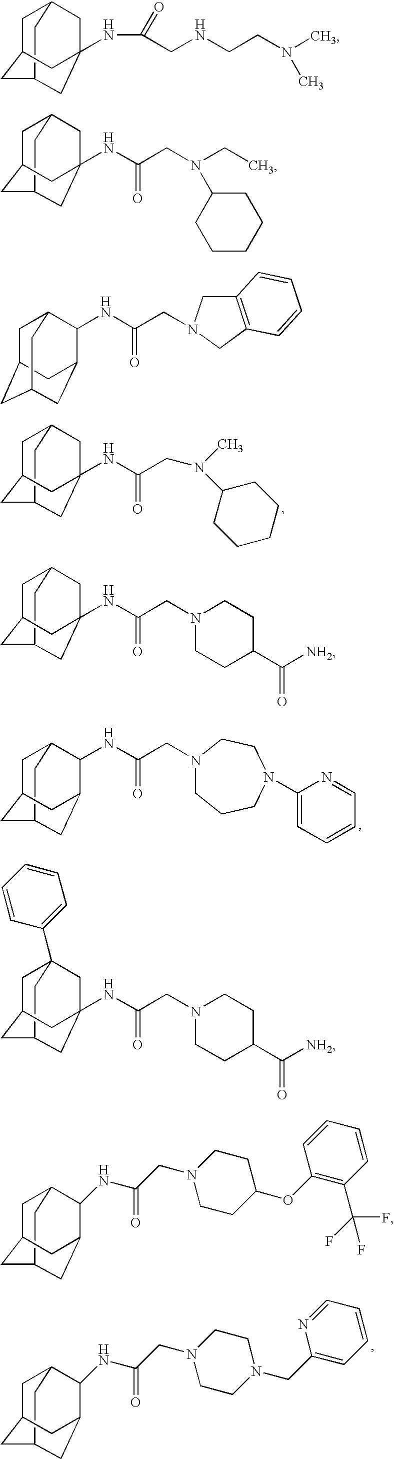

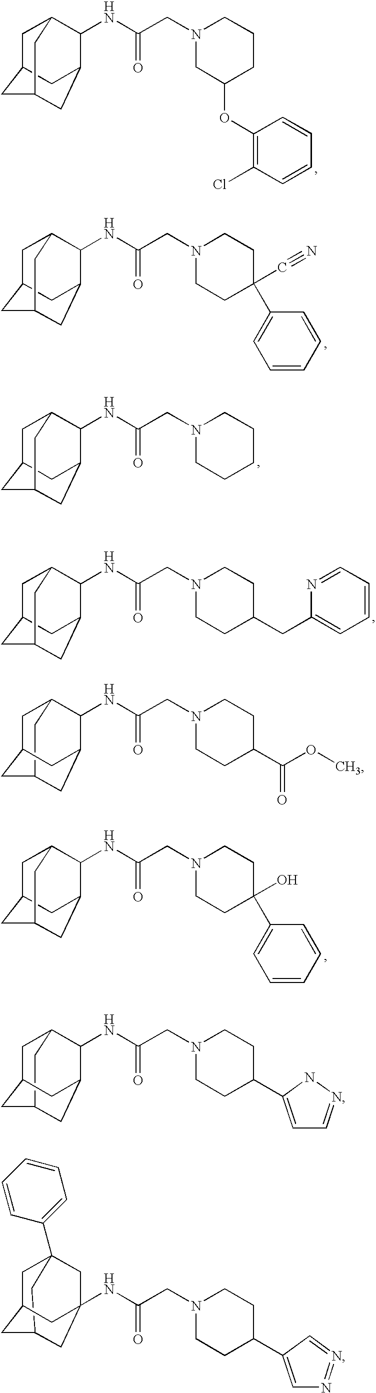

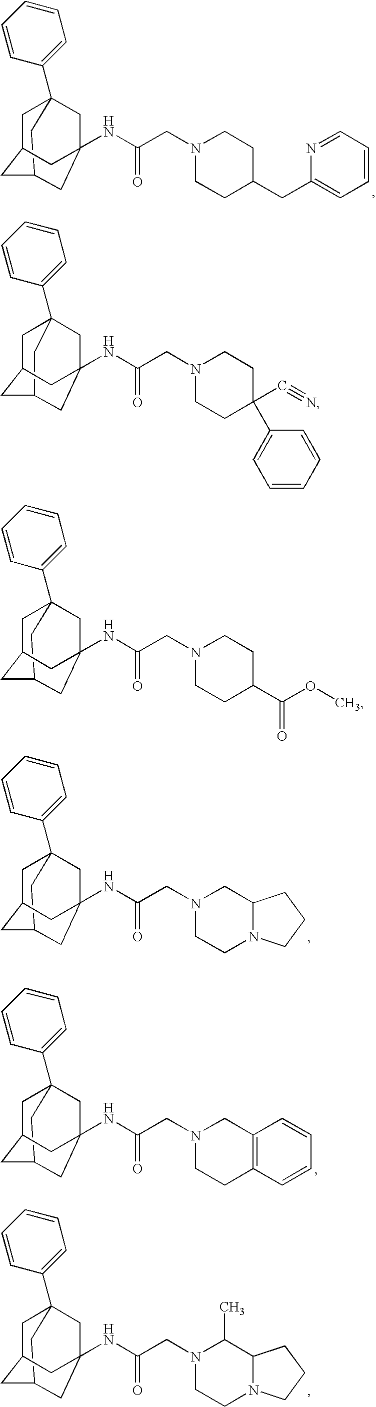

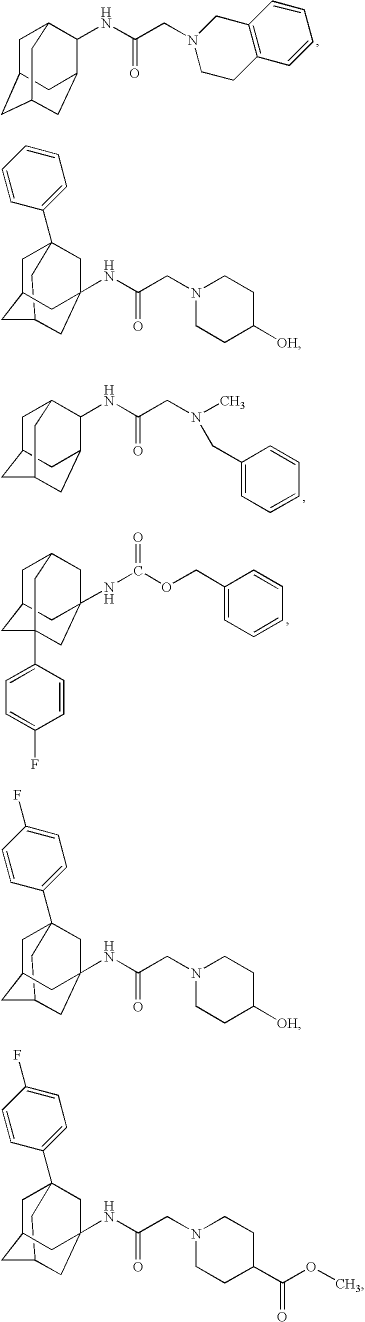

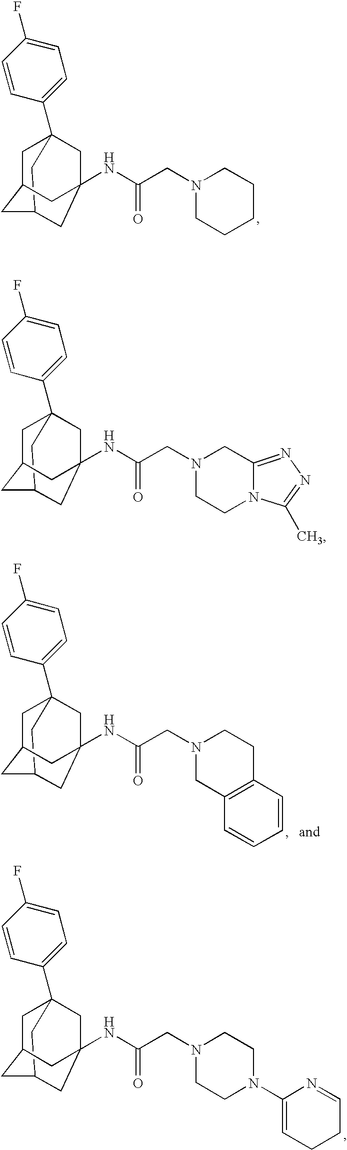

or pharmaceutically acceptable salts thereof.

An embodiment of the invention according to a compound of formula (I) is selected from the group consisting of:

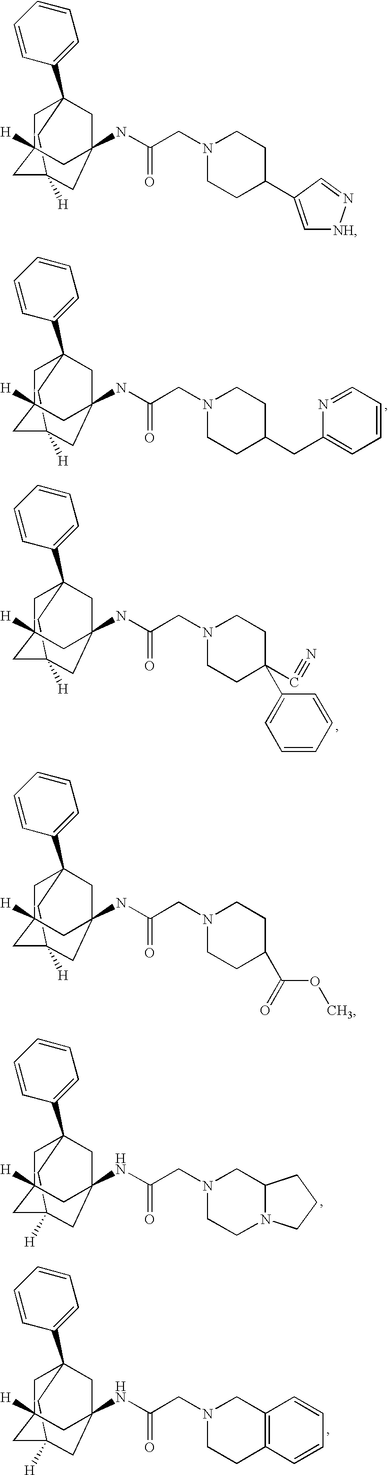

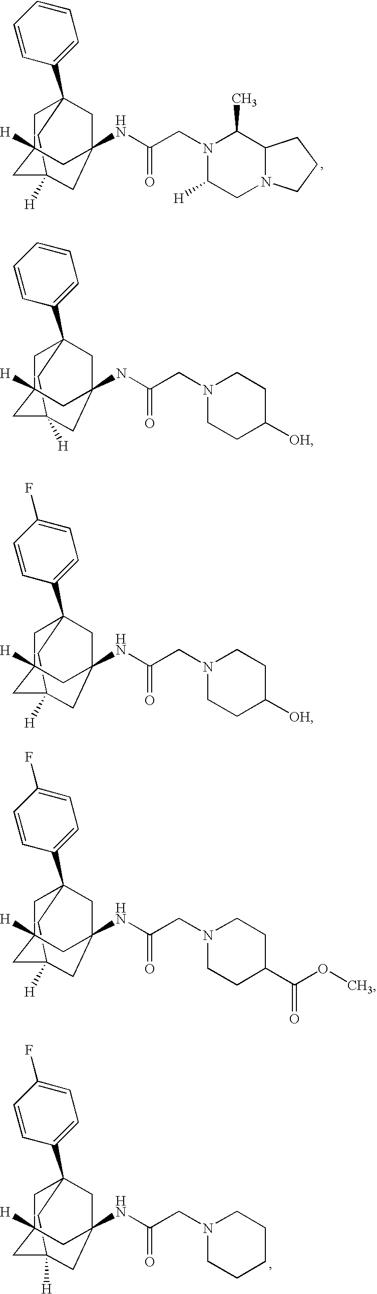

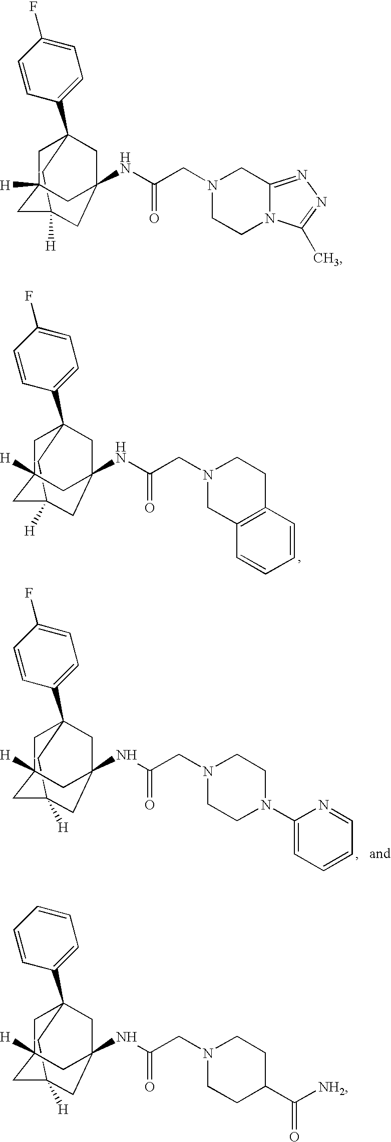

or pharmaceutically acceptable salts thereof.

In an embodiment of the invention, a pharmaceutical composition comprising an effective amount of a compound according to formula (I), or a pharmaceutically acceptable salt or solvate thereof, and a pharmaceutically acceptable carrier.

An yet another embodiment of the invention, a method of treating a condition that is mediated by the modulation of the 11-β-hsd-1 enzyme, the method comprising administering to a mammal an effective amount of a compound according to formula (I), or a pharmaceutically acceptable salt or solvate thereof.

In yet another embodiment of the invention, a method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which liver is a target organ, the method comprising administering to a mammal an effective amount of a compound according to formula (I), or a pharmaceutically acceptable salt or solvate thereof.

In another embodiment of the invention, a method of treating a condition that is mediated by the modulation of the 11-β-hsd-1 enzyme, the method comprising administering to a mammal an effective amount of a compound according to formula (I), in combination further comprising a therapeutic agent to treat glaucoma, or a pharmaceutically acceptable salt or solvate thereof.

In yet another embodiment, the method of treating a condition comprising administering to a mammal an effective amount of a compound according to formula (I), in combination with a prostanoid receptor agonist, wherein said agonist is lantanoprost, to treat glaucoma, or a pharmaceutically acceptable salt or solvate thereof.

In yet another embodiment of the invention, the method of treating a condition comprising administering to a mammal an effective amount of a compound according to formula (I), in combination with a known therapeutic agent, wherein said agent is a carbonic anhydrase inhibitor, to treat glaucoma or a pharmaceutically acceptable salt or solvate thereof.

In an embodiment of the invention, the method of treating a condition comprising administering to a mammal an effective amount of a compound according to formula (I), in combination with a known therapeutic agent, wherein said therapeutic agent is a PPAR agonist.

In an embodiment of the invention, a method of preparing a compound of formula (III)

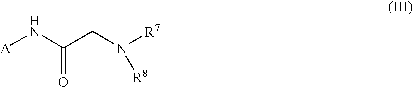

wherein:

R⁷ and R⁸ are independently selected from the group consisting of H, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl; or

R⁷ and R⁸ may optionally be taken together with the nitrogen to which they are attached to form a (4-11)-membered heterocyclic which may be fused or unfused;

t is selected from the group consisting of 0, 1, 2, 3, 4, and 5;

each R⁹ and R¹⁰ is independently selected from the group consisting of H, (C₁-C₆) alkyl, (C₂-C₆) alkenyl, and (C₂-C₆) alkynyl;

A is adamantyl, unsubstituted or substituted with 1 to 5 substituents independently selected from the group consisting of halo, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl;

comprising the steps of:

treating a compound of formula (II)

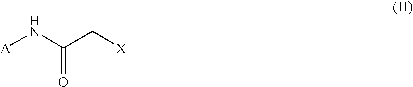

wherein:

X is a leaving group; and

A is defined as above;

with an amine in a solvent in the presence of a base to produce a compound of formula (III); and treating a compound of formula (Ia)

A-NH₂ (Ia)

wherein:

A is defined as above;

with an acyl halide in a solvent in the presence of a base to produce a compound of formula (II).

In yet another embodiment, the method wherein X in step (a) is selected from group consisting of Cl, Br, and methanesulfonate.

In yet another embodiment, the method wherein the amine in step (a), is R⁷R⁸NH.

In another embodiment, the method wherein the base in step (a) is selected from the group consisting of K₂CO₃, NaHCO₃, and (C₂H₅)₃N.

In yet another embodiment, the method wherein step (a) proceeds at a temperature range from about 20 degrees Celsius to the boiling point of the solvent.

In yet another embodiment, the method wherein the solvent in step (b) is CH₂Cl₂ or acetonitrile.

In yet another embodiment, the method wherein the base in step (b) is (C₂H₅)₃N or NaHCO₃.

In yet another embodiment, the method wherein step (b) proceeds at a temperature range from about −15 degrees Celsius to about 50 degrees Celsius.

In an embodiment of the invention, a method of preparing a compound of formula (IIa)

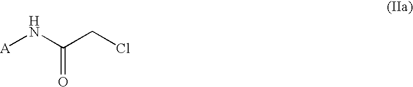

wherein:

A is adamantyl, unsubstituted or substituted with 1 to 5 substituents independently selected from the group consisting of halo, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl;

each R⁹ and R¹⁰ is independently selected from the group consisting of H, (C₁-C₆) alkyl, (C₂-C₆) alkenyl, and (C₂-C₆) alkynyl;

t is selected from the group consisting of 0, 1, 2, 3, 4, and 5;

comprising the steps of:

(c) treating a compound of formula (V)

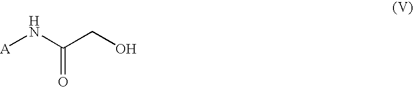

wherein:

A is defined as above;

with neat SOCl₂ or SOCl₂ in a solvent to form a compound of formula (IIa);

(d) treating a compound of formula (IV)

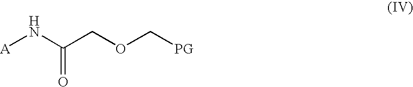

wherein:

A is defined as above;

PG is protecting group;

with a protecting group removing agent in a solvent to form a compound of formula (V);

and

(e) coupling a compound of formula (Ia) A-NH₂

A-NH₂ (Ia)

wherein:

A is defined as above;

with an acid to form a compound of formula (IV).

In yet another embodiment, the method wherein the solvent in step (c) is CCl₄.

In yet another embodiment, the method wherein step (c) is performed at a temperature from 20 degrees Celsius to 100 degrees Celsius.

In another embodiment, the method wherein PG in step (d) is (C₆-C₁₂) aryl.

In yet another embodiment, the method wherein PG is phenyl.

In yet another embodiment, the method wherein the protecting group removing agent in step (d) is (CH₃)₃Sil.

In yet another embodiment, the method wherein the solvent in step (d) is CHCl₃.

In yet another embodiment, the method wherein step (d) is performed at a temperature from 20 degrees Celsius to the boiling point of the solvent.

In yet another embodiment, the method wherein the acid in step (e) is benzyloxyacetic acid.

In an embodiment of the invention, a method of preparing a compound of formula (III)

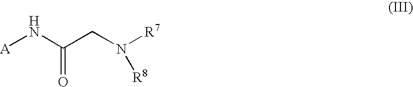

wherein:

R⁷ and R⁸ are independently selected from the group consisting of H, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl; or

R⁷ and R⁸ may optionally be taken together with the nitrogen to which they are attached to form a (4-11) membered heterocyclic which may be fused or unfused;

each R⁹ and R¹⁰ is independently selected from the group consisting of H, (C₁-C₆) alkyl, (C₂-C₆) alkenyl, and (C₂-C₆) alkynyl;

A is adamantyl, unsubstituted or substituted with 1 to 5 substituents independently selected from the group consisting of halo, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl;

t is selected from the group consisting of 0, 1, 2, 3, 4, and 5;

comprising the steps of:

(f) treating a compound of formula (Ia) A-NH₂ with a compound of formula (VI)

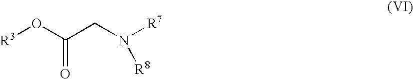

wherein:

R³ is (C₁-C₆) alkyl;

R⁷ and R⁸ are defined above;

in the presence of a reagent in a suitable solvent to form a compound of formula (III);

(g) treating a compound of formula (VII)

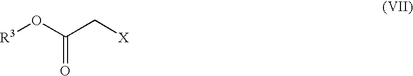

wherein:

R³ is defined above;

X is a leaving group;

with an amine in a suitable solvent in the presence of a base to form a compound of formula (VI).

In yet another embodiment, the method wherein in step (f) R³ is methyl or ethyl.

In yet another embodiment, the method wherein X in step (f) is selected from the group consisting of Cl, Br and methanesulfonate.

In yet another embodiment, the method wherein step (f) is performed with the reagent Al(CH₃)₂Cl.

In yet another embodiment, the method wherein step (f) is performed with the solvent CH₂Cl₂.

In yet another embodiment, the method wherein step (f) is performed at a temperature from 0 degrees Celsius to about 20 degrees Celsius.

In yet another embodiment, the method wherein the amine in step (g) is R⁷R⁸NH.

In yet another embodiment, the method wherein the solvent in step (g) is CH₂Cl₂ or DMF.

In yet another embodiment, the method wherein the base in step (g) is NaHCO₃ or triethylamine.

### DEFINITIONS

For purposes of the present invention, as described and claimed herein, the following terms are defined as follows:

As used herein, the terms “comprising” and “including” are used in their open, non-limiting sense.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term, “OMs” as used herein, is intended to mean, unless otherwise indicated methanesulfonate.

The term “Me” as used herein, unless otherwise indicated, is intended to mean means methyl.

The term “Et” as used herein, unless otherwise indicated, is intended to mean means ethyl.

The term “Et₃N” as used herein, unless otherwise indicated, is intended to mean means triethylamine.

The term “EtOAc” as used herein, unless otherwise indicated, is ethyl acetate.

The term “AlMe₂Cl” as used herein, unless otherwise indicated, is intended to mean dimethyl aluminum chloride.

The term “Ac” as used herein, unless otherwise indicated, is intended to mean means acetyl.

The term “NT”, as used herein, unless otherwise indicated, is intended to mean not tested.

The term “TFA” as used herein, unless otherwise indicated, is intended to mean trifluoroacetic acid.

The term TEA, as used herein, unless otherwise indicated, is intended to mean triethanolamine.

The term G6P, as used herein, unless otherwise indicated, is intended to mean glucose-6-phosphate.

The term NADPH, as used herein, unless otherwise indicated, is intended to mean nicotinamide adenine dinucleotide phosphate, reduced form.

In accordance convention, in some structural formula herein, the carbon atoms and their bound hydrogen atoms are not explicitly depicted e.g., [symbol] represents a methyl group,

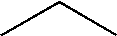

represents an ethyl group,

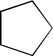

represents a cyclopentyl group, etc.

The term “amino”, as used herein, is intended to include the —NH₂ radical, and any substitutions of the N atom.

The terms “halogen” and “halo,” as used herein represent chlorine, fluorine, bromine or iodine.

The term “trifluoromethyl,” as used herein, is meant to represent a —CF₃ group.

The term “trifluoromethoxy,” as used herein, is meant to represent a —OCF₃ group.

The term “cyano,” as used herein, is meant to represent a —CN group.

The term “substituted,” means that the specified group or moiety bears one or more substituents. The term “unsubstituted,” means that the specified group bears no substituents. The term “optionally substituted” means that the specified group is unsubstituted or substituted by one or more substituents.

The term “neat” as used herein, is meant to represent an absence of solvent.

Terms such as —(CR⁷R⁸)ₜ or —(CR¹⁴R¹⁵)ᵥ, for example, are used, R⁷, R⁸, R¹⁴ and R¹⁵ may vary with each iteration of t or v above 1. For instance, where t or v is 2 the terms —(CR⁷R⁸)ᵥ or —(CR¹⁴R¹⁵)ₜ may equal —CH₂CH₂—, or —CH(CH₃)C(CH₂CH₃)(CH₂CH₂CH₃)—, or any number of similar moieties falling within the scope of the definitions of R⁷, R⁸, R¹⁴ and R¹⁵.

The term Ki, as used herein, is intended to mean values of enzyme inhibition constant.

The term IC₅₀, as used herein, is intended to mean concentrations required for 50% enzyme inhibition.

The term “min”, as used herein, is intended to mean, unless otherwise indicated, minutes.

The term “m/z”, as used herein, is intended to mean, unless otherwise indicated, mass/charge ratio.

The term “DMF”, as used herein, is intended to mean, unless otherwise indicated, N,N-dimethylformamide.

The term “DMSO”, as used herein, is intended to mean, unless otherwise indicated refers to dimethylsulfoxide.

The phrase “pharmaceutically acceptable salt(s)”, as used herein, unless otherwise indicated, includes salts of acidic or basic groups which may be present in the compounds of formula (I). The compounds of formula (I) that are basic in nature are capable of forming a wide variety of salts with various inorganic and organic acids. The acids that may be used to prepare pharmaceutically acceptable acid addition salts of such basic compounds of formula (I) are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts.

The term “diseases in which the liver is a target organ”, as used herein, unless otherwise indicated means diabetes, hepatitis, liver cancer, liver fibrosis, and malaria.

The term “metabolic syndrome”, as used herein, unless otherwise indicated means psoriasis, diabetes mellitus, wound healing, inflammation, neurodegenerative diseases, galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m², and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels”, as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being preferred. As noted previously, since the activity of different compounds which fall within the definition of Formula (I) as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, non insulin dependent diabetes mellitus (NIDDM), arterial hypertension, central (visceral) obesity, and dyslipidemia.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-10 carbon atoms, such as cyclopropyl, cyclobutyl, cyclopentyl, cyclohexyl, cycloheptyl, and adamantyl. Illustrative examples of cycloalkyl are derived from, but not limited to, the following:

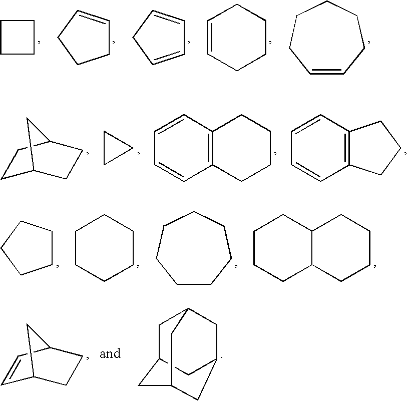

The term “aryl”, as used herein, unless otherwise indicated, includes an organic radical derived from an aromatic hydrocarbon by removal of one hydrogen, such as phenyl or naphthyl.

The term “(3-7)-membered heterocyclyl”, “(6-10)-membered heterocyclyl”, or “(4-11)-membered heterocyclyl”, as used herein, unless otherwise indicated, includes aromatic and non-aromatic heterocyclic groups containing one to four heteroatoms each selected from O, S and N, wherein each heterocyclic group has from 3-7, 6-10, or 4-11 atoms, respectively, in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3 membered heterocyclic group is aziridine, an example of a 4 membered heterocyclic group is azetidinyl (derived from azetidine). An example of a 5 membered heterocyclic group is thiazolyl, an example of a 7 membered ring is azepinyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-7 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-7 membered heterocyclic are derived from, but not limited to, the following:

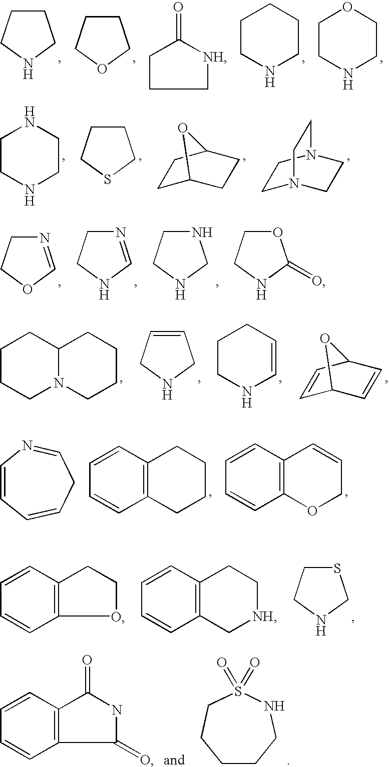

Unless otherwise indicated, the term “oxo” refers to ═O.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO (dimethylsulfoxide), ethyl acetate, acetic acid, or ethanolamine.

The compounds of the present invention may have asymmetric carbon atoms. The carbon-carbon bonds of the compounds of the present invention may be depicted herein using a solid line (—), a solid wedge ( [symbol] ), or a dotted wedge ( [symbol] ). The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Solutions of individual stereoisomeric compounds of the present invention may rotate plane-polarized light. The use of either a “(+)” or “(−)” symbol in the name of a compound of the invention indicates that a solution of a particular stereoisomer rotates plane-polarized light in the (+) or (−) direction, as measured using techniques known to those of ordinary skill in the art.

Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

Alternatively, individual stereoisomeric compounds of the present invention may be prepared in enantiomerically enriched form by asymmetric synthesis. Asymmetric synthesis may be performed using techniques known to those of skill in the art, such as the use of asymmetric starting materials that are commercially available or readily prepared using methods known to those of ordinary skill in the art, the use of asymmetric auxiliaries that may be removed at the completion of the synthesis, or the resolution of intermediate compounds using enzymatic methods. The choice of such a method will depend on factors that include, but are not limited to, the availability of starting materials, the relative efficiency of a method, and whether such methods are useful for the compounds of the invention containing particular functional groups. Such choices are within the knowledge of one of ordinary skill in the art.

When the compounds of the present invention contain asymmetric carbon atoms, the derivative salts, prodrugs and solvates may exist as single stereoisomers, racemates, and/or mixtures of enantiomers and/or diastereomers. All such single stereoisomers, racemates, and mixtures thereof are intended to be within the scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of derivatives, prodrugs, salts, or solvates that are solids, it is understood by those skilled in the art that the derivatives, prodrugs, salts, and solvates used in the method of the invention, may exist in different polymorph or crystal forms, all of which are intended to be within the scope of the present invention and specified formulas. In addition, the derivative, salts, prodrugs and solvates used in the method of the invention may exist as tautomers, all of which are intended to be within the broad scope of the present invention.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Certain compounds of formula (I) may have asymmetric centers and therefore exist in different enantiomeric forms. All optical isomers and stereoisomers of the compounds of formula (I), and mixtures thereof, are considered to be within the scope of the invention. With respect to the compounds of formula (I), the invention includes the use of a racemate, one or more enantiomeric forms, one or more diastereomeric forms, or mixtures thereof. The compounds of formula (I) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts or solvates of said compounds which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) of this invention thereof can generally be prepared by carrying out the procedures disclosed in the Schemes and/or in the Examples below, by substituting a readily available isotopically labeled reagent for a non-isotopically labeled reagent.

Other aspects, advantages, and features of the invention will become apparent from the detailed description below.

### DETAILED DESCRIPTION AND EMBODIMENTS OF THE INVENTION

The following reaction Schemes illustrate the preparation of the compounds of the present invention. Unless otherwise indicated, R¹-R²², in the reaction schemes and the discussion that follow are as defined above.

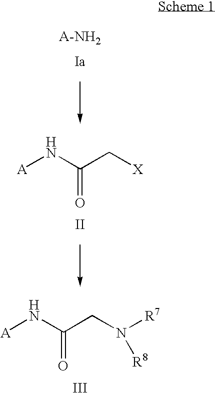

Referring to Scheme 1 above, the compound of formula III may be prepared by reacting a compound of formula II with an amine, R⁷R⁸NH, wherein the group X is a leaving group such as Cl, Br, OMs, etc., and R⁷ and R⁸ are independently selected from the group consisting of H, (C₁-C₆) alkyl, (C₂-C₆) alkenyl, (C₂-C₆) alkynyl —(CR⁷R⁸)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl, or R⁷ and R⁸ can form a (4-11)-membered heterocyclic which may be fused or unfused and each R⁹ and R¹⁰ is independently selected from the group consisting of H, (C₁-C₆) alkyl, (C₂-C₆) alkenyl, and (C₂-C₆) alkynyl, in a suitable solvent (e.g. dichloromethane or DMF) advantageously, in the presence of a base (e.g. K₂CO₃, NaHCO₃, triethylamine), at a suitable temperature ranging from about room temperature to the boiling point of the solvent, typically from about 20 degrees Celsius to about 100 degrees Celsius. The compound of formula II may be prepared by reacting a compound of formula Ia with XCH₂COCl, wherein X is Cl or Br, in a suitable solvent such as CH₂Cl₂ and acetonitrile, in the presence of a base such as NaHCO₃ or triethylamine at a suitable temperature ranging from −15 degrees Celsius to 50 degrees Celsius. Compound of formula Ia, which is commercially available to one of skill in the art, is an amine wherein A is adamantyl, unsubstituted or substituted with 1 to 5 substituents independently selected from the group consisting of halo, (C₁-C₆) alkyl, —CF₃, —CN, and phenyl, etc.

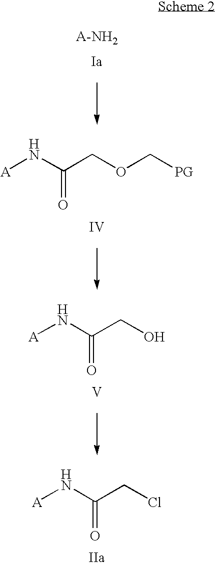

Referring to Scheme 2, compound of formula IIa may also be prepared as illustrated in Scheme 2, by reacting a compound of formula V with neat SOCl₂ or SOCl₂ in a suitable solvent such as CCl₄, at a suitable temperature ranging from room temperature to 100 degrees Celsius. The compound of formula V may be prepared by reacting a compound of formula IV, wherein PG is a suitable protecting group such as phenyl, with (CH)₃Sil in a suitable solvent such as CHCl₃ at a suitable temperature from room temperature to the boiling point of the solvent, typically from about 20 degrees Celsius to about 100 degrees Celsius. The compound of formula IV may be prepared by coupling the compound of formula Ia, which is commercially available to one of skill in the art, is an amine wherein A is adamantyl, unsubstituted or substituted with 1 to 5 substituents independently selected from the group consisting of halo, (C₁-C₆) alkyl, —CF₃, —CN, and phenyl, etc., with a suitable acid such as benzyloxyacetic acid by a method known to those skilled in the art.

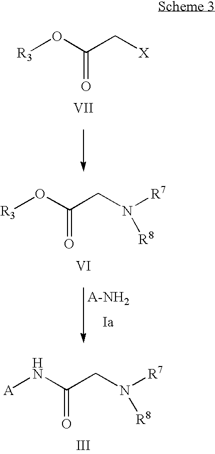

Referring to Scheme 3, the compound of formula III may also be prepared by reacting a compound of formula VI with a compound of formula Ia in the presence of a reagent such as Al(CH₃)₂Cl in a suitable solvent such as CH₂Cl₂ at a suitable temperature from 0 degrees Celsius to room temperature. The compound of formula VI may be prepared by reacting a compound of formula VII with an amine with a formula of R⁷R⁸NH, wherein the group X is a leaving group such as Cl, Br, OMs, etc., and R⁷ and R⁸ are independently selected from the group consisting of H, (C₁-C₆) alkyl, (C₂-C₆) alkenyl, (C₂-C₆) alkynyl —(CR⁷R⁸)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl, or R⁷ and R⁸ can form a (4-11)-membered heterocyclic which may be fused or unfused and each R⁹ and R¹⁰ is independently selected from the group consisting of H and (C₁-C₆) alkyl, in a suitable solvent such as CH₂Cl₂ or DMF in the presence of a base such as NaHCO₃ or triethylamine, at a suitable temperature from 0 degrees Celsius to 100 degrees Celsius. R³ is (C₁-C₆) alkyl, suitably methyl or ethyl.

All starting materials, regents, and solvents are commercially available and are known to those of skill in the art unless otherwise stated. These chemical manipulations are known to those skilled in the art and include (a) removal of a protecting group by methods outlined in T. W. Greene and P. G. M. Wuts, “Protective Groups in Organic Synthesis”, Second Edition, John Wiley and Sons, New York, 1991; (b) displacement of a leaving group (halide, mesylate, tosylate, etc) with a primary or secondary amine, thiol or alcohol to form a secondary or tertiary amine, thioether or ether, respectively; (c) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; (d) reductive amination of a primary or secondary amine using an aldehyde.

The compounds of the present invention may have asymmetric carbon atoms. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formula (I) that are basic in nature are capable of forming a wide variety of different salts with various inorganic and organic acids. Although such salts must be pharmaceutically acceptable for administration to animals, it is often desirable in practice to initially isolate the compound of formula (I) from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula (I) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula (I). Such non-toxic base salts include those derived from such pharmacologically acceptable cations as sodium, potassium, calcium, and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention may be modulators of 11-β-hsd-1. The compounds of the present invention may modulate processes mediated by 11-β-hsd-1, which refer to biological, physiological, endocrinological, and other bodily processes which are mediated by receptor or receptor combinations which are responsive to the 11-β-hsd-1 inhibitors described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g.) brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such processes can be accomplished in vitro or in vivo. In vivo modulation can be carried out in a wide range of subjects, such as, for example, humans, rodents, sheep, pigs, cows, and the like.

The compounds according to the present invention may be used in several indications which involve modulations of 11-β-hsd-1 enzyme. Thus, the compounds according to the present invention may be used against dementia (see WO97/07789), osteoporosis (see Canalis E, Mechanisms of glucocorticoid action in bone: implications to glucocorticoid-induced osteoporosis, Journal of Clinical Endocrinology and Metabolism, 1996, 81, 3441-3447) and may also be used regarding disorders of the immune system (see Franchimont, et al, “Inhibition of Th1 immune response by glucocorticoids: dexamethasone selectively inhibits IL-12-induced Stat 4 phosphorylation in T lymphocytes”, The Journal of Immunology 2000, Feb. 15, vol. 164 (4), pages 1768-74) and also in the above listed indications.

Inhibition of 11-β-hsd-1 in mature adipocytes is expected to attenuate secretion of the plasminogen activator inhibitor 1 (PAI-1) an independent cardiovascular risk factor (Halleux, C. M., et al. (1999) J. Clin. Endocrinol. Metab. 84: 4097-4105). Furthermore, there is a clear correlation between glucocorticoid “activity” and cardiovascular risk factor suggesting that a reduction of the glucocorticoid effects would be beneficial (Walker, B. R., et al. (1998) Hypertension 31: 891-895; Fraser, R., et al. (1999) Hypertension 33: 1364-1368).

Adrenalectomy attenuates the effect of fasting to increase both food intake and hypothalamic neuropeptide Y expression. This supports the role of glucocorticoids in promoting food intake and suggests that inhibition of 11-β-hsd-1 in the brain might increase satiety and therefore reduce food intake (Woods, S. C. et al. (1998) Science, 280: 1378-1383).

Inhibition of 11-β-hsd-1 in isolated murine pancreatic 1-cells improves the glucose-stimulated insulin secretion (Davani, B. et al. (2000) J. Biol. Chem. Nov. 10, 2000; 275(45): 34841-4). Glucocorticoids were previously known to reduce pancreatic insulin release in vivo (Billaudel, B. and B. C. J. Sutter (1979) Horm. Metab. Res. 11: 555-560). Thus, inhibition of 11-β-hsd-1 is predicted to yield other beneficial effects for diabetes treatment, besides effects on liver and fat.

Stress and glucocorticoids influence cognitive function (de Quervain, D. J.-F., B. Roozendaal, and J. L. McGaugh (1998) Nature 394: 787-790). The enzyme 11-β-hsd-1 controls the level of glucocorticoid action in the brain and thus contributes to neurotoxicity (Rajan, V., C. R. W. Edwards, and J. R. Secki, J. (1996) Neuroscience 16: 65-70; Seckl, J. R., Front. (2000) Neuroendocrinol. 18: 49-99). Unpublished results indicate significant memory improvement in rats treated with a non-specific 11-β-hsd-1 inhibitor. Based the above and on the known effects of glucocorticoids in the brain, it may also be suggested that inhibiting 11-β-hsd-1 in the brain may result in reduced anxiety (Tronche, F. et al. (1999) Nature Genetics 23: 99-10:3). Thus, taken together, the hypothesis is that inhibition of 11-β-hsd-1 in the human brain would prevent reactivation of cortisone into cortisol and protect against deleterious glucocorticoid-mediated effects on neuronal survival and other aspects of neuronal function, including cognitive impairment, depression, and increased appetite.

The general perception is that glucocorticoids suppress the immune system. But in fact there is a dynamic interaction between the immune system and the HPA (hypothalamo-pituitary-adrenal) axis (Rook, G. A. W. (1999) Baillier's Clin. Endocrinol. Metab. 13: 576-581). The balance between the cell-mediated response and humoral responses is modulated by glucocorticoids. A high glucocorticoid activity, such as at a state of stress, is associated with a humoral response. Thus, inhibition of the enzyme 11-β-hsd-1 has been suggested as a means of shifting the response towards a cell-based reaction.

Recent data suggests that the levels of the glucocorticoid target receptors and the 11-β-hsd-1 enzymes determine the susceptibility to glaucoma (Stokes, J. et al. (2000) Invest. Opthalmol. 41: 1629-1638). Further, inhibition of 11-β-hsd-1 was recently presented as a novel approach to lower the intraocular pressure (Walker E. A. et al, poster P3-698 at the Endocrine society meeting Jun. 12-15, 1999, San Diego). Ingestion of carbenoxolone, a non-specific inhibitor of 11-β-hsd-1, was shown to reduce the intraocular pressure by 20% in normal subjects. In the eye, expression of 11-β-hsd-1 is confined to basal cells of the corneal epithelium and the non-pigmented epithelialium of the cornea (the site of aqueous production), to ciliary muscle and to the sphincter and dilator muscles of the iris. In contrast, the distant isoenzyme 11 beta-hydroxysteroid dehydrogenase type 2 is highly expressed in the non-pigmented ciliary epithelium and corneal endothelium. None of the enzymes is found at the trabecular meshwork, the site of drainage. Thus, 11-β-hsd-1 is suggested to have a role in aqueous production, rather than drainage, but it is presently unknown if this is by interfering with activation of the glucocorticoid or the mineralocorticoid receptor, or both.

Glucocorticoids have an essential role in skeletal development and function but are detrimental in excess (Kim, C. H., S. L. Cheng, and G. S. Kim (1999) J. Endocrinol. 162: 371-379). The negative effect on bone nodule formation could be blocked by the non-specific inhibitor carbenoxolone suggesting an important role of 11-β-hsd-1 in the glucocorticoid effect (Bellows, C. G., A. Ciaccia, and J. N. M. Heersche, (1998) Bone 23: 119-125). Other data suggest a role of 11-β-hsd-1 in providing sufficiently high levels of active glucocorticoid in osteoclasts, and thus in augmenting bone resorption (Cooper, M. S. et al. (2000) Bone 27: 375-381). Taken together, these different data suggest that inhibition of 11-β-hsd-1 may have beneficial effects against osteoporosis by more than one mechanism working in parallel.

Bile acids inhibit 11β-hydroxysteroid dehydrogenase type 2. This results in a shift in the overall body balance in favor of cortisol over cortisone, as shown by studying the ratio of the urinary metabolites (Quattropani C, Vogt B, Odermatt A, Dick B, Frey B M, Frey F J. 2001. J Clin Invest. November; 108(9):1299-305. “Reduced activity of 11beta-hydroxysteroid dehydrogenase in patients with cholestasis”). Reducing the activity of 11-β-hsd-1 in the liver by a selective inhibitor is predicted to reverse this imbalance, and acutely counter the symptoms such as hypertension, while awaiting surgical treatment removing the biliary obstruction.

The compounds of the present invention may also be useful in the treatment of other metabolic disorders associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and many other conditions linked to NIDDM, including dyslipidemia glucocorticoid induced insulin resistance, dyslipidemia, polycystic ovarian syndrome, obesity, hyperglycemia, hyperlipidemia, hypercholesteremia, hypertriglyceridemia, hyperinsulinemia, and hypertension. Brief definitions of these conditions are available in any medical dictionary, for instance, Stedman's Medical Dictionary (10^{th} Ed.).

### Assay

All assays were performed using an Agilent 1100 HPLC (High Performance Liquid Chromatograph) with a 96-well plate autosampler accompanied by an IN/US systems β-Ram model 3 Radio-HPLC detector. The 11β-hsd-1 assay was performed on a Corning® 96-well plate at a total volume of 300 microliters. The buffer conditions used in this assay are as follows: 100 millimolar TEA, 200 millimolar NaCl, 0.02% n-dodecyl beta-D-maltoside (NDM), 5% glycerol, 5 millimolar β-ME, at a pH of 8.0. The reaction mixture for the assay includes 500 micromolar NADPH, about 6 nanomolar 11β-hsd-1 (based on active site titration with potent reversible inhibitor), 1% DMSO (inhibitor), 2 millimolar G6P, 1 U/milliliter G6P dehydrogenase, and 6 millimolar MgCl₂. G6P, G6P dehydrogenase and MgCl₂ act as a regeneration system to amplify 11β-hsd-1 activity. NADPH and 11β-hsd-1 were incubated in buffer for 30 minutes in the presence of inhibitor at 25 Celsius prior to the addition of the regeneration system and initiation with ³H-cortisone.

Initial reaction velocities were measured by stopping the reaction at various time points between 0 and 60 minutes by mixing 60 microliters of sample with 60 microliters of DMSO. These samples were then analyzed by reversed phase liquid chromatography by injecting 15 microliters of sample into a Jupiter C18, 150×4.6 millimeters, 5 micron, 300 Angstrom Phenomenex® column, commercially available from Phenomenex of Torrance, Calif., while running an isocratic method of 34:66 methanol to water at 1.25 milliliters/minute. The β-Ram model 3 pumps at a 3:1 liquid scintillation cocktail to eluate ratio, and a ³H signal is subsequently measured by the area of the peak observed. ³H-cortisone comes off at approximately 7 minutes, while the ³H-cortisol product of the 11β-hsd-1 reaction comes off at approximately 9 minutes. The area of ³H-cortisol is then plotted versus time to determine a linear velocity and this rate can then be plotted to inhibitor concentration to determine a Kᵢ and IC₅₀.

[1,2-3H]-cortisone is commercially available from American Radiolabeled Chemicals Inc. of St. Louis, Mo. NADPH, (G6P), and Glucose-6-Phosphate dehydrogenase are commercially available from Sigma, Ronkonkoma, N.Y. United States.

The Kᵢ values of the compounds of the present invention for the 11-β-hsd-1 enzyme may lie typically between about 10 nano molar and about 10 micro molar. The compounds of the present invention that were tested all have Kᵢ's in at least one of the above SPA assays of less than 1 micro molar, preferably less than 100 nano molar. Certain preferred groups of compounds possess differential selectivity toward the various 11-β-hsd's. One group of preferred compounds possesses selective activity towards 11-β-hsd-1 over 11β-hsd-2. Another preferred group of compounds possesses selective activity towards 11β hsd-2 over 11-β-hsd-1.

### Pharmaceutical Compositions/Formulations, Dosaging and Modes of Administration

Methods of preparing various pharmaceutical compositions with a specific amount of active compound are known, or will be apparent, to those skilled in this art. In addition, those of ordinary skill in the art are familiar with formulation and administration techniques. Such topics would be discussed, e.g. in Goodman and Gilman's The Pharmaceutical Basis of Therapeutics, current edition, Pergamon Press; and Remington's Pharmaceutical Sciences, current edition. Mack Publishing, Co., Easton, Pa. These techniques can be employed in appropriate aspects and embodiments of the methods and compositions described herein. The following examples are provided for illustrative purposes only and are not meant to serve as limitations of the present invention.

The compounds of formula (I) may be provided in suitable topical, oral and parenteral pharmaceutical formulations for use in the treatment of 11-β-hsd-1 mediated diseases.

The compounds of the present invention may be administered orally as tablets or capsules, as oily or aqueous suspensions, lozenges, troches, powders, granules, emulsions, syrups or elixirs.

The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occurring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to know methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the acceptable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The compounds of formula (I) may also be administered in the form of suppositories for rectal administration of the drug. These compositions can be prepared by mixing the drug with a suitable non-irritating excipient that is solid at about 25 Celcius but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The compounds of formula (I) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. A preferred dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered concomitantly with other orally active antidiabetic compounds such as the sulfonylureas, for example, tolbutamide and the like.

### EXAMPLES

The examples, general procedures, and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Specific Examples of various compounds according to the invention may be advantageously prepared as set out in the Examples below. The structures of the following Example were confirmed by one or more of the following: proton magnetic resonance spectroscopy (NMR), infrared spectroscopy (IR), elemental microanalysis, atmospheric pressure chemical ionization mass spectroscopy (APCI-MS), thin layer chromatography (TLC), melting point (MP), boiling point (BP), and high performance liquid chromatography (HPLC).

Where HPLC chromatography is referred to in the preparations and examples below, the general conditions used, unless otherwise indicated, are as follows. The column used is an Alltech Platinum EPS 100 Angstrom 1.5 micron C18 column; 33 millimeter×7 millimeter. The samples are run on a Hewlett Packard-1100 system. A gradient solvent method is used running 5% acetonitrile in water (0.1% trifluoroacetic acid) to 95% acetonitrile in water (0.1% trifluoroacetic acid) over 5.5 minutes. The system then proceeds on a wash cycle with 95 percent acetonitrile in water (0.1% trifluoroacetic acid) for 1.5 minutes. The flow rate over this period is a constant 1.5 milliliters/minute.

The structures of the compounds are confirmed by either elemental analysis or NMR, where peaks assigned to the characteristic protons in the titled compound are presented where appropriate. ¹H NMR shift (δ_{H}) are given in parts per million (ppm) down filed from an internal reference standard.

The invention will now be described in reference to the following EXAMPLES. These EXAMPLES are not to be regarded as limiting the scope of the present invention, but shall only serve in an illustrative manner.

### Method A

### Example 1 N-1-Adamantyl-2-[4-(2-methoxyphenoxy)piperidin-1-yl]acetamide

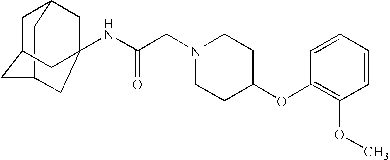

To a 2 neck round bottom flask, which had been oven-dried and N₂ cooled, was added 1-adamantamine (437 milligrams) and anhydrous CH₂Cl₂ (12 milliliters). To this solution at 0 degrees Celsius under N₂ was added a 1 molar solution of Al(CH₃)₂Cl in hexane (2.9 milliliters) portion wise. After stirring the cloudy reaction mixture at 0 degrees Celsius for 30 minutes and room temperature for 1.7 hours a solution of ethyl [4-(2-methoxyphenoxy)piperidin-1-yl]acetate (170 milligrams) in anhydrous CH₂Cl₂ was added slowly. After stirring at room temperature for 43 hours, the reaction mixture was cooled to 0 degrees Celsius and quenched with water (10 milliliters) drop wise, diluted with CH₂Cl₂ (9 milliliters). The layer was separated and the aqueous layer was extracted with CH₂Cl₂ (4×10 milliliters). The combined organic layers were dried with MgSO₄. The solvent was removed in vacuo, and the residue was purified using reversed phase Kromasil® C18, 0.05% TFA in water and acetonitrile to provide the titled product as a TFA salt (6.9 milligrams) with retention time of 14.8 minutes APCIMS: m/z 399.3 (M+1).

### Example 2 2-(4-Acetyl-1,4-diazepan-1-yl)-N-1-adamantylacetamide

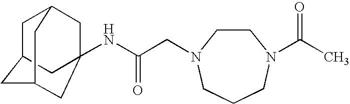

The titled compound was prepared analogously to Example 1 using instead 1-adamantamine (792 milligrams) and ethyl (4-acetyl-1,4-diazepan-1-yl)acetate (410 milligrams) to provide the titled product as a TFA salt (14.7 milligrams) with retention time of 10.8 min. APCIMS: m/z 334.3 (M+1).

### Example 3 N-(1-Adamantylmethyl)-2-[4-(2-methoxyphenoxy)piperidin-1-yl]acetamide

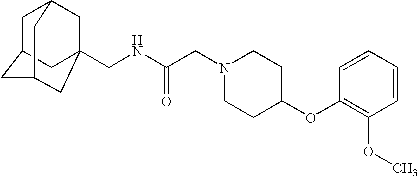

To The titled compound was prepared analogously to Example 1 using instead 1-adamantanmethyl amine (498 milligrams) and ethyl [4-(2-methoxyphenoxy)piperidin-1-yl]acetate (177 milligrams) to provide the titled product as a TFA salt (57 milligrams) with retention time of 15.5 min. APCIMS: m/z 413.3 (M+1).

### Example 4 N¹-1-Adamantyl-N²-cyclohexyl-N²-ethylglycinamide

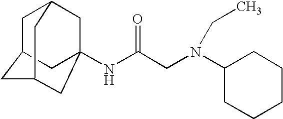

The titled compound was prepared analogously to Example 1 using instead 1-adamantanamine (711 milligrams) and ethyl N-cyclohexyl-N-ethylglycinate (500 milligrams) to provide the titled product as a TFA salt (43 milligrams) with retention time of 14.6 min. APCIMS: m/z 319.3 (M+1).

### Preparation 4a Ethyl N-cyclohexyl-N-ethylglycinate

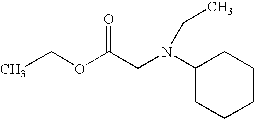

Ethyl bromoacetate (608 milligrams), N-ethylcyclohexylamine (507 milligrams), K₂CO₃ (2.57 g), water (10 milliliter) were combined at room temperature. After stirring at 60° C. for 20 hours, the reaction mixture was cooled to R.T. and diluted with water (10.0 milliliter) and extracted with CH₂Cl₂ (4× to a total volume of 100 milliliter). The combined organic layers were dried with K₂CO₃. The solvent was removed in vacuo to provide the titled product (720 milligrams). APCIMS: m/z 214.4 (M+1).

### Example 5 N¹-(1-Adamantylmethyl)-N²-(3,3,5-trimethylcyclohexyl)glycinamide

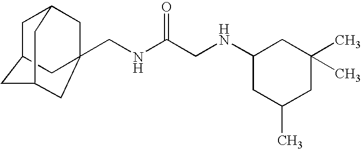

The titled compound was prepared analogously to Example 1 using instead 1-adamantanmethyl amine (260 milligrams) and ethyl N-(3,3,5-trimethylcyclohexyl)glycinate (178 milligrams) to provide the titled product as a TFA salt (160 milligrams) with retention time of 16.9 min. APCIMS: m/z 347.3 (M+1). ¹H NMR (400 MHz, CHLOROFORM-D) δ ppm 0.53 (1H, q, J=11.96 Hz) 0.72-0.79 (1H, m) 0.84-0.89 (6H, m) 0.90-0.94 (3H, m) 1.27-1.37 (3H, m) 1.43-1.51 (6H, m) 1.53-1.64 (5H, m) 1.65-1.75 (3H, m) 1.85-1.94 (1H, m) 1.94-2.02 (3H, m) 2.54 (1H, ft, J=11.62, 3.79 Hz) 2.94 (2H, qd, J=13.31, 6.57 Hz) 3.25-3.30 (2H, m) 7.53 (1H, s).

### Example 6 N¹-1-Adamantyl-N²-(3,3,5-trimethylcyclohexyl)glycinamide

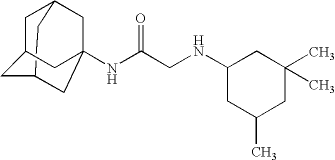

The titled compound was prepared analogously to Example 1 using instead 1-adamantanamine (620 milligrams) and ethyl N-(3,3,5-trimethylcyclohexyl)glycinate (307 milligrams) to provide the titled product as a TFA salt (45 milligrams) with retention time of 16 min. APCIMS: m/z 333.3 (M+1).

### Example 7 N¹-2-Adamantyl-N²-(3,3,5-trimethylcyclohexyl)glycinamide

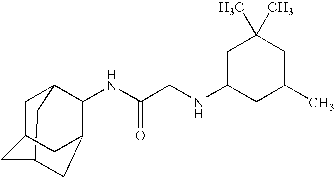

The titled compound was prepared analogously to Example 1 using instead 2-adamantanamine HCl (414 milligrams) and ethyl N-(3,3,5-trimethylcyclohexyl)glycinate (100 milligrams) to provide the titled product as a TFA salt (37 milligrams) with retention time of 16 minutes. APCIMS: m/z 333.3 (M+1). ¹H NMR (400 MHz, MeOD) δ ppm 0.84-0.90 (2H, m) 0.95-1.05 (11H, m) 1.18 (1H, t, J=12.38 Hz) 1.44 (2H, dt, J=13.39, 1.77 Hz) 1.65-2.07 (15H, m) 3.80-3.89 (2H, m) 4.03 (1H, s).

### Example 8 N²-Adamantyl-2-[4-(2-methoxyphenoxy)piperidin-1-yl]acetamide

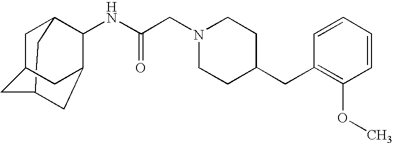

The titled compound was prepared analogously to Example 1 using instead 2-adamantylamine HCl and (400 milligrams), and N-1-adamantyl-2-[4-(2-methoxyphenoxy)piperidin-1-yl]acetamide, to provide the titled product as a TFA salt (68.5 milligrams) with retention time of 14.7 minutes. APCIMS: m/z 399.3 (M+1) and as a free base (13.8 milligrams). ¹H NMR (400 MHz, MeOD) δ ppm 1.68 (2H, d, J=12.38 Hz) 1.75-1.87 (14H, m) 1.91-2.00 (2H, m) 2.34-2.44 (2H, m) 2.74-2.84 (2H, m) 3.00 (2H, s) 3.26 (1H, dt, J=3.28, 1.64 Hz) 3.77 (3H, s) 3.96 (1H, s) 4.22-4.31 (1H, m) 6.82 (1H, td, J=7.33, 2.02 Hz) 6.87-6.94 (3H, m).

### Example 9 N¹-2-Adamantyl-N²-cyclohexyl-N²-methylglycinamide

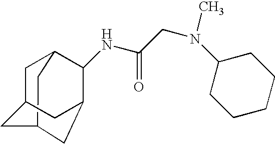

The titled compound was prepared analogously to Example 1 using instead ethyl N-cyclohexyl-N-methylglycinate (300 milligrams) and 2-adamantamine HCl (860 milligrams), to provide the titled product as a TFA salt (360.7 milligrams) with a retention time of 13.2 minutes. APCIMS: m/z 305.5 (M+1). ¹H NMR (400 MHz, MeOD) δ ppm 1.16-1.27 (1H, m) 1.35-1.52 (3H, m) 1.63-1.74 (3H, m) 1.80 (2H, s) 1.83-1.99 (14H, m) 2.07-2.13 (1H, m) 2.84 (3H, s) 3.23 (1H, tt, J=11.91, 3.38 Hz) 3.77-3.87 (1H, m) 4.02-4.12 (2H, m) 8.24 (1H, s).

### Preparation 9a Ethyl N-cyclohexyl-N-methylglycinate

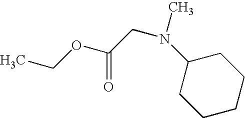

The titled compound was prepared analogously to Example 4a using instead ethyl bromoacetate (503 milligrams), N-methylcyclohexylamine (386 milligrams), K₂CO₃ (2.06 g), water (8 milliliter) as an oil (488 milligrams). APCIMS: m/z 214.4 (M+1).

### Example 10 N¹-2-Adamantyl-N²-cyclohexyl-N²-ethylglycinamide

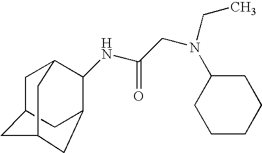

The titled compound was prepared analogously to Example 1 using instead ethyl N-cyclohexyl-N-ethylglycinate (303 milligrams), and 2-adamantamine HCl (799 milligrams), to provide the titled product as a TFA salt (310.1 milligrams) with a retention time of 13.9 minutes. APCIMS: m/z 319.5 (M+1). ¹H NMR (400 MHz, MeOD) δ ppm 1.26-1.34 (3H, m) 1.36-1.47 (4H, m) 1.63-1.74 (3H, m) 1.80-1.99 (16H, m) 2.04-2.13 (1H, m) 3.24-3.29 (3H, m) 3.79 (1H, d, J=16.17 Hz) 4.01-4.12 (2H, m) 8.25 (1H, s).

### Example 11 N²-Adamantyl-2-(1,3-dihydro-2H-isoindol-2-yl)acetamide

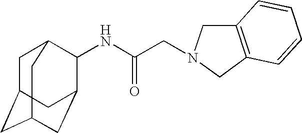

The titled compound was prepared analogously to Example 1 using instead ethyl 1,3-dihydro-2H-isoindol-2-ylacetate (352 milligrams) and 2-adamantamine.HCl (982 milligrams) to provide the titled product as a TFA salt (303.2 milligrams) with a retention time of 13.23 minutes. APCIMS: m/z 311.5 (M+1). ¹H NMR (400 MHz, MeOD) δ ppm 1.49 (2H, d, J=12.63 Hz) 1.58-1.77 (14H, m) 3.10-3.20 (1H, m) 3.83-3.91 (5H, m) 6.99-7.08 (4H, m).

### Preparation 11a Ethyl 1,3-dihydro-2H-isoindol-2-ylacetate

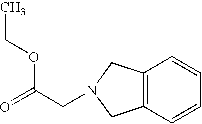

### Method B

### Example 12 N-1-Adamantyl-2-(benzyloxy)acetamide

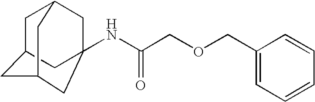

To a vial containing (benzyloxy)acetyl chloride (406 milligrams) was added 1-adamantanamine (983 milligrams) followed with anhydrous DMF (2 milliliter). After heating in microwave for 50 mins at 80° C., the reaction mixture was quenched with water (10 milliliter) and extracted with CH₂Cl₂ (4×10 milliliter). The combined organic layers were dried with MgSO₄. The solvent was removed in vacuo, and the residue was purified using reversed phase Kromasil C18, 0.05% TFA in water and acetonitrile to provide the titled product as a TFA salt (22 milligrams) with retention time of 20.5 min. APCIMS: m/z 300.1 (M+1) Kᵢ:NT.

### Method C

### Example 13 N-(1-Adamantylmethyl)-2-(4,5,7,8-tetrahydro-6H-isoxazolo[3,4-d]azepin-6-yl)acetamide

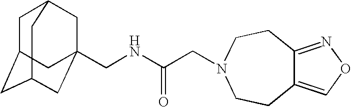

N-(1-adamantylmethyl)-2-chloroacetamide (58.6 milligrams), 5,6,7,8-tetrahydro-4H-isoxazolo[3,4-d]azepine (25.3 milligrams), K₂CO₃ (75.1 milligrams) and CH₂Cl₂ (3) were combined at room temperature. The reaction flask was capped with a yellow stopper and after stirring at 40° C. for 19 hours; the reaction mixture was stopped, cooled to room temperature, and quenched with water (4 milliliters). The layer was separated and the aqueous layer was extracted with EtOAc (3×5 milliliters). The combined organic layers were dried with K₂CO₃. The solvent was removed in vacuo, and the residue was purified using reversed phase Kromasil C18, 0.05% TFA in water and acetonitrile to provide the titled product as a TFA salt (16.2 milligrams) with retention time of 12.9. APCIMS: m/z 344.3 (M+1) Kᵢ:NT.

### Example 14 Ethyl 4-{[2-(1-adamantylamino)-2-oxoethyl]amino}piperidine-1-carboxylate

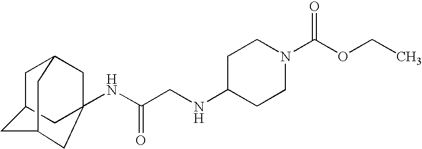

The titled compound was prepared analogously to Example 12 using instead N-1-adamantyl-2-chloroacetamide (250 milligrams), ethyl-4-amino-1-piperidinecarboxylate (191 milligrams), K₂CO₃ (450 milligrams) and CH₂Cl₂ (10 milliliter) as TFA salt (170 milligrams) with retention time of 12.5. APCIMS: m/z 364.3 (M+1).

### Preparation 14a N-1-Adamantyl-2-chloroacetamide

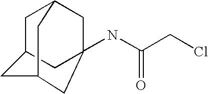

The titled compound was prepared analogously to Example 1 using instead 1-adamataneamine (8.8 g) in methylene chloride (150 milliliter) followed with K₂CO₃ (21.98 g) at 0° C. under nitrogen. After stirring at 0° C. for 20 mins and at R.T for 17.5 hours, methylene chloride (50 milliliter) was added. After stirring at R.T for 5 hours the reaction mixture was diluted with water (150 milliliter) and extracted with methylene chloride (2×70 milliliter). The aqueous layer was diluted with water (50 milliliter) and extracted with methylene chloride (3×70 milliliter). The combined organic layers were dried with K₂CO₃. The solvent was removed in vacuo to give the desired product as a white solid (11 g).

### Example 15 N-1-Adamantyl-2-[4-(1,3-benzodioxol-5-ylmethyl)piperazin-1-yl]acetamide

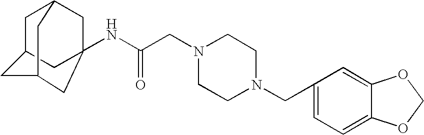

The titled compound was prepared analogously to Example 12 using instead N-1-adamantyl-2-chloroacetamide (202 milligrams), 1-(1,3-benzodioxol-5-ylmethyl)piperazine (232 milligrams), K₂CO₃ (373 milligrams) and CH₂Cl₂ (3 milliliter) as TFA salt (192.4 milligrams) with retention time of 12.7. APCIMS: m/z 412.3 (M+1).

### Example 16 Ethyl 4-({2-[(1-adamantylmethyl)amino]-2-oxoethyl}amino)piperidine-1-carboxylate

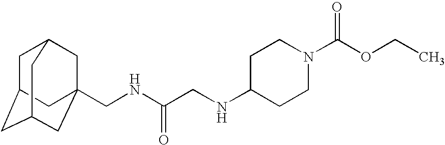

The titled compound was prepared analogously to Example 12 using instead N-(1-adamantylmethyl)-2-chloroacetamide (100 milligrams), ethyl-1-piperazinecarboxylate (75 milligrams), K₂CO₃ (172 milligrams) and CH₂Cl₂ (2 milliliter) as TFA salt (18.7 milligrams) with retention time of 13.5. APCIMS: m/z 378.3 (M+1).

### Preparation 16a N-(1-Adamantylmethyl)-2-chloroacetamide

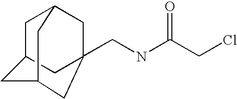

To a flask containing chloroacetyl chloride (350 milligrams) in anhydrous methylene chloride (6 milliliter) was added a solution of 1-adamatane methyl amine (500 milligrams) in methylene chloride followed with K₂CO₃ at 0° C. under nitrogen. After stirring at 0° C. for 20 mins and at R.T for 16 hours, the reaction mixture was diluted with water (5 milliliter) and extracted with methylene chloride (10 milliliter×4). The combined organic layers were dried with MgSO₄. The solvent was removed in vacuo to give the desired product as a liquid (800 milligrams contaminated with trace of 1-adamatane methyl amine).

### Example 17 N-(1-Adamantylmethyl)-2-[4-(1,3-benzodioxol-5-ylmethyl)piperazin-1-yl]acetamide

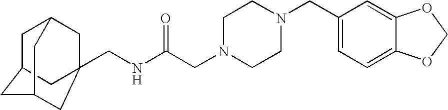

The titled compound was prepared analogously to Example 12 using instead N-(1-adamantylmethyl)-2-chloroacetamide (100 milligrams), 1-(1,3-benzodioxol-5-ylmethyl)piperazine (101 milligrams), K₂CO₃ (170 milligrams) and CH₂Cl₂ 3 milliliter) as TFA salt (28 milligrams) with retention time of 13.8. APCIMS: m/z 426.3 (M+1).

### Example 18 2-(4-Acetyl-1,4-diazepan-1-yl)-N-(1-adamantylmethyl)acetamide

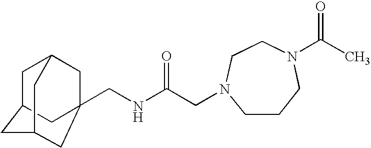

The titled compound was prepared analogously to Example 12 using instead N-(1-adamantylmethyl)-2-chloroacetamide (172 milligrams), N-acetylhomopiperazine (156 milligrams), K₂CO₃ (352 milligrams) and CH₂Cl₂ (6 milliliter) as TFA salt (64 milligrams) with retention time of 11.8. APCIMS: m/z 348.3 (M+1).

### Method D

### Example 19 N-(1-Adamantyl)-2-[4-(hydroxymethyl)piperidin-1-yl]acetamide

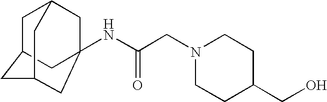

N-(1-adamantyl)-2-chloroacetamide (190 milligrams), 4-piperidine methanol (92 milligrams), K₂CO₃ (219 milligrams) and DMSO (2 milliliter) were combined at room temperature. After stirring at 40° C. for 15 hours, the reaction mixture was stopped, cooled to room temperature, filtered, and the residue was purified using reversed phase Kromasil C18, 0.05% TFA in water and acetonitrile to provide the titled product as a TFA salt (182.3 milligrams) with retention time of 10.8 min. APCIMS: m/z 307.3 (M+1). Kᵢ:NT.

### Example 20 N¹-1-Adamantyl-N²-(pyridin-2-ylmethyl)glycinamide

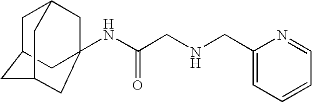

The titled compound was prepared analogously to Example 19 using instead N-1-adamantyl-2-chloroacetamide (136 milligrams) and 2-(aminomethyl)pyridine (81 milligrams), to provide the titled compound as a TFA salt (92.4 milligrams) with retention time of 12 min. APCIMS: m/z 300.2 (M+1).

### Example 21 N-1-Adamantyl-2-(4-pyridin-2-ylpiperazin-1-yl)acetamide

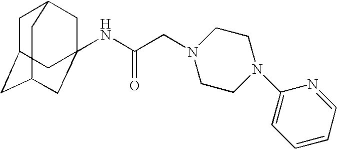

The titled compound was prepared analogously to Example 19 using instead N-1-adamantyl-2-chloroacetamide (136 milligrams) and 1-(2-pyridyl)piperazine (123 milligrams) to provide the titled compound as a TFA salt (116.4 milligrams) with retention time of 10.1 minutes. APCIMS: m/z 355.3 (M+1).

### Example 22 N-2-Adamantyl-2-[3-(2-chlorophenoxy)piperidin-1-yl]acetamide

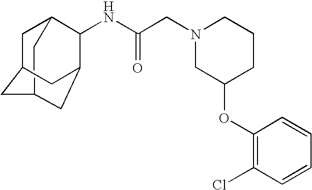

The titled compound was prepared analogously to Example 19 using instead N-2-adamantyl-2-chloroacetamide (152 milligrams), 4-(2-trifluoromethylphenoxy)piperidine HCl (142 milligrams), to provide the titled compound as a TFA salt (107.6 milligrams) with retention time of 15.9 minutes. APCIMS: m/z 403.2 (M+1).

### Preparation 22a N-2-Adamantyl-2-chloroacetamide

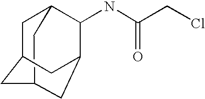

The titled compound was prepared analogously to Preparation 14a using instead chloroacetyl chloride (1.4 g), anhydrous methylene chloride (60 milliliter), 2-adamantylamine (1.865 g), K₂CO₃ (5.09 g) to produce a white solid (2.33 g).

### Example 23 N-2-Adamantyl-2-(4-cyano-4-phenylpiperidin-1-yl)acetamide

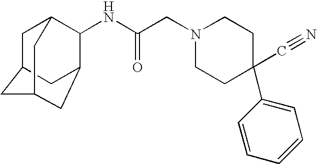

The titled compound was prepared analogously to Example 19 using instead N-2-adamantyl-2-chloroacetamide (98.6 milligrams) and 4-cyano-4-phenyl piperidine HCl (104 milligrams), to provide the titled compound as a TFA salt (59.8 milligrams) with retention time of 14.5 minutes. APCIMS: m/z 378.5 (M+1).

### Example 24 N-2-Adamantyl-2-piperidin-1-ylacetamide

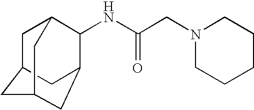

The titled compound was prepared analogously to Example 19 using instead N-2-adamantyl-2-chloroacetamide (101.6 milligrams) and piperidine (52 milligrams), to provide the titled compound as a TFA salt (83.3 milligrams) with retention time of 11.6 minutes. APCIMS: m/z 277.4 (M+1). ¹H NMR (400 MHz, MeOD) δ ppm 1.49-1.58 (1H, m) 1.64 (2H, d, J=12.88 Hz) 1.78-2.00 (17H, m) 3.02 (2H, t, J=111.49 Hz) 3.53 (2H, d, J=11.62 Hz) 3.93 (2H, s) 4.03 (1H, s) 8.16 (1H, s).

### Example 25 N-2-Adamantyl-2-[4-(pyridin-2-ylmethyl)piperidin-1-yl]acetamide

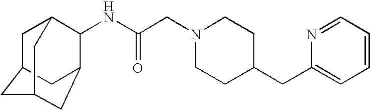

The titled compound was prepared analogously to Example 19 using instead N-2-adamantyl-2-chloroacetamide (152 milligrams) and 2-piperidine-4-ylmethylpyridine 2HCl (182 milligrams) to provide the titled compound as a TFA salt (151.2 milligrams) with retention time of 9.8 minutes. APCIMS: m/z 368.5 (M+1). ¹H NMR (400 MHz, MeOD) δ ppm 1.60-1.92 (19H, m) 2.17 (1H, s) 3.06 (3H, d, J=6.32 Hz) 3.59 (2H, s) 3.94-4.07 (3H, m) 7.84-7.94 (2H, m) 8.47 (1H, td, J=7.89, 1.64 Hz) 8.76 (1H, d, J=5.05 Hz).

### Example 26 Methyl 1-[2-(2-adamantylamino)-2-oxoethyl]piperidine-4-carboxylate

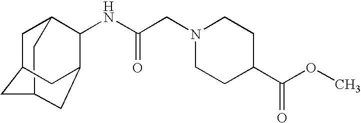

The titled compound was prepared analogously to Example 19 using instead N-2-adamantyl-2-chloroacetamide (108 milligrams) and methyl isonipecotate (102 milligrams), to provide the titled compound as a TFA salt (92.6 milligrams) with retention time of 12.1 minutes APCIMS: m/z 335.3 (M+1).

### Example 27 N-2-Adamantyl-2-(4-hydroxy-4-phenylpiperidin-1-yl)acetamide

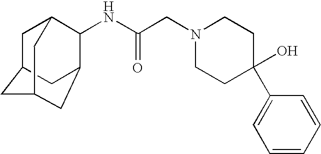

The titled compound was prepared analogously to Example 19 using instead N-2-adamantyl-2-chloroacetamide (110 milligrams) and 4-hydroxyl-4-phenylpiperidine (103.4 milligrams) to provide the titled compound as a TFA salt (105.8 milligrams) with retention time of 13.4 minutes APCIMS: m/z 369.5 (M+1).

### Example 28 N-2-Adamantyl-2-[4-(1H-pyrazol-5-yl)piperidin-1-yl]acetamide

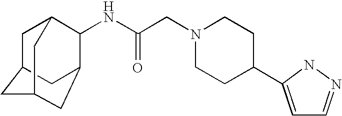

The titled compound was prepared analogously to Example 19 using instead N-2-adamantyl-2-chloroacetamide (99 milligrams) and 4-(1H-pyrazol-5-yl)piperidine (73 milligrams), to provide the titled compound as a TFA salt (77.3 milligrams) with retention time of 11.3 minutes APCIMS: m/z 343.3 (M+1).

### Example 29 N-[(5R,7S)-3-phenyl-1-adamantyl]-2-[4-(1H-pyrazol-4-yl)piperidin-1-yl]acetamide

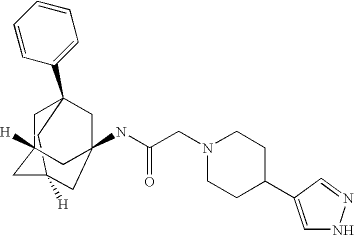

The titled compound was prepared analogously to Example 19 using instead 2-chloro-N-(3-phenyl-1-adamantyl)acetamide (170 milligrams) and 4-(1H-pyrazol-5-yl)piperidine (84 milligrams), to provide the titled compound as a TFA salt (20.3 milligrams) with retention time of 14.3 minutes APCIMS: m/z 419.6 (M+1). ¹H NMR (400 MHz, MeOD) δ ppm 1.75 (2H, s) 1.89 (4H, d, J=1.77 Hz) 2.01-2.12 (6H, m) 2.16-2.22 (4H, m) 2.27 (3H, s) 2.94-3.04 (1H, m) 3.11-3.21 (2H, m) 3.60-3.69 (2H, m) 3.83 (2H, s) 6.21 (1H, s) 7.10-7.17 (1H, m) 7.25-7.36 (4H, m) 7.57 (1H, d, J=2.27 Hz) 7.95 (1H, s).

### Preparation 29a Benzyl (3-phenyl-1-adamantyl)carbamate

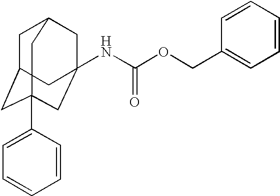

To a flask containing 3-phenyladamantane-1-carboxylic acid (649 milligrams) was added diphenylphosphorylazide (772 milligrams), toluene (6 milliliter), Et₃N (0.4 milliliter) at R.T. After stirring at 70° C. for approximately 1.2 hours, benzyl alcohol (0.28 milliliter) was added to the reaction mixture. After stirring at 70° C. for 19.4 hours, the reaction mixture was cooled to R.T and the solvents were removed under reduced pressure. The obtained residue was diluted in EtOAc (10 milliliter) and water (5 milliliter). The layers were separated. The aqueous layer was extracted with EtOAc (2×10 milliliter). The combined organic layers were dried with MilligramsSO₄, concentrated under reduced pressure to provide the desired product (700 milligrams)

### Preparation 29b (3-Phenyl-1-adamantyl)amine

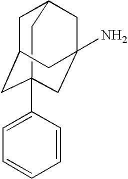

To a solution of benzyl (3-phenyl-1-adamantyl)carbamate (320 milligrams) in EtOAc (3 milliliter) was added 10% Pd/C (330 milligrams) and acetic acid. After hydrogenated over night at R.T., the reaction mixture was diluted with EtOAc and filtered. The solvents were removed under reduced pressure to give an oil that solidified at R.T. to provide the desired product (177 milligrams). APCIMS: m/z 227.2 (M+1).

### Preparation 29c 2-Chloro-N-(3-phenyl-1-adamantyl)acetamide

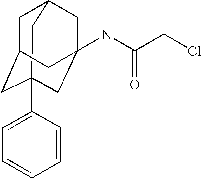

The titled compound was prepared analogously to Preparation 14a using instead chloroacetyl chloride (97.4 milligrams), anhydrous methylene chloride (3.5 milliliter), (3-phenyl-1-adamantyl)amine (177 milligrams), K₂CO₃ (324 milligrams) and weighed 160 milligrams.

### Example 30 N-[(5R,7S)-3-phenyl-1-adamantyl]-2-[4-(pyridin-2-ylmethyl)piperidin-1-yl]acetamide

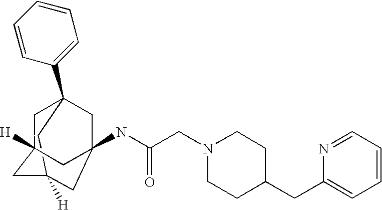

The titled compound was prepared analogously to Example 19 using instead 2-chloro-N-(3-phenyl-1-adamantyl)acetamide (147.9 milligrams) and 2-piperidin-4-ylmethylpiperidine 2HCl (138.2 milligrams) to provide the titled compound as a TFA salt (26.8 milligrams) with retention time of 12.5 minutes APCIMS: m/z 444.3 (M+1). ¹H NMR (400 MHz, MeOD) δ ppm 1.64-1.75 (4H, m) 1.84-1.94 (7H, m) 2.01-2.28 (8H, m) 3.01 (4H, d, J=5.81 Hz) 3.55 (2H, s) 3.79 (2H, s) 7.12-7.35 (5H, m) 7.77-7.85 (2H, m) 7.95 (1H, m) 8.37 (1H, td, J=7.89, 1.64 Hz) 8.70 (1H, d, J=5.05 Hz).

### Example 31 2-(4-cyano-4-phenylpiperidin-1-yl)-N-[(5R,7S)-3-phenyl-1-adamantyl]acetamide

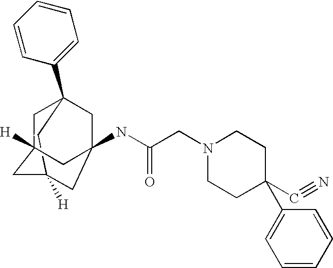

The titled compound was prepared analogously to Example 19 using instead 2-chloro-N-(3-phenyl-1-adamantyl)acetamide (150 milligrams), 4-cyano-4-phenylpiperidine HCl (122 milligrams), to provide the titled compound as a TFA salt (24.5 milligrams) with retention time of 17.2 minutes APCIMS: m/z 454.6 (M+1). ¹H NMR (400 MHz, MeOD) δ ppm 1.76 (2H, s) 1.86-1.95 (4H, m) 2.03-2.15 (5H, m) 2.20 (2H, s) 2.28 (2H, s) 2.48 (2H, s) 2.50 (2H, d, J=2.53 Hz) 3.42-3.53 (2H, m) 3.72-3.84 (2H, m) 3.99 (2H, s) 7.15 (1H, t, J=7.20 Hz) 7.25-7.32 (2H, m) 7.33-7.38 (2H, m) 7.39-7.45 (1H, m) 7.45-7.51 (2H, m) 7.54-7.59 (2H, m).

### Example 32 Methyl 1-(2-oxo-2-{[(5R,7S)-3-phenyl-1-adamantyl]amino}ethyl)piperidine-4-carboxylate

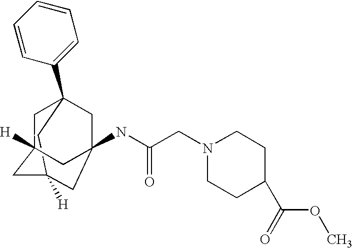

The titled compound was prepared analogously to Example 19 using instead 2-chloro-N-(3-phenyl-1-adamantyl)acetamide (150 milligrams) and methyl isonipecotate (78 milligrams) to provide the titled compound as a free base after treating the TFA salt with MP-CO₃ and weighed 11.3 milligrams with retention time of 15.2 minutes APCIMS: m/z 411.6 (M+1). ¹H NMR (400 MHz, MeOD) δ ppm 1.63-1.74 (4H, m) 1.81-1.90 (6H, m) 1.96-2.06 (4H, m) 2.11 (2H, s) 2.13-2.24 (5H, m) 2.26-2.35 (1H, m) 2.75-2.82 (2H, m) 2.85 (2H, s) 3.57-3.64 (3H, m) 7.07-7.13 (1H, m) 7.21-7.27 (2H, m) 7.29-7.34 (2H, m).

### Example 33 2-(Hexahydropyrrolo[1,2-a]pyrazin-2(1H)-yl)-N-[(5R,7S)-3-phenyl-1-adamantyl]acetamide

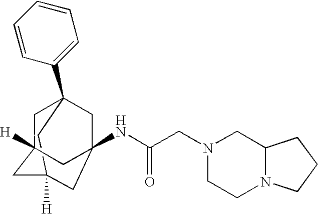

The titled compound was prepared analogously to Example 19 using instead 2-chloro-N-(3-phenyl-1-adamantyl)acetamide (156 milligrams) and octahydropyrrolo[1,2-a]pyrazine (69 milligrams), to provide the titled compound as a TFA salt (22 milligrams) with retention time of 13.6 minutes APCIMS: m/z 419.6 (M+1).

### Example 34 2-(3,4-dihydroisoquinolin-2(1H)-yl)-N-[(5R,7S)-3-phenyl-1-adamantyl]acetamide

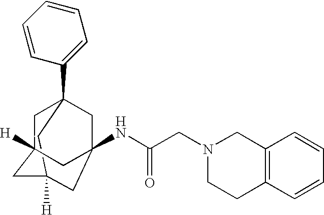

The titled compound was prepared analogously to Example 19 using instead 2-chloro-N-(3-phenyl-1-adamantyl)acetamide (156 milligrams) and 1,2,3,4-tetrahydroisoquinoline (270 milligrams), to provide the titled compound as a TFA salt (20.9 milligrams) with retention time of 16.6 minutes. APCIMS: m/z 401.6 (M+1).

### Example 35 2-[(3S,8aS)-3-Methylhexahydropyrrolo[1,2-a]pyrazin-2(1H)-yl]-N-[(5R,7S)-3-phenyl-1-adamantyl]acetamide

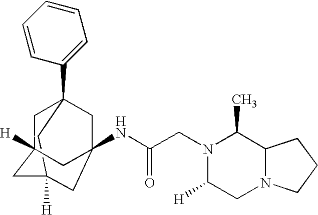

The titled compound was prepared analogously to Example 19 using instead 2-chloro-N-(3-phenyl-1-adamantyl)acetamide (155 milligrams) and 3-methyloctahydropyrrolo[1,2-a]pyrazine (88 milligrams), to provide the titled compound as a TFA salt (15.5 milligrams) with retention time of 14.7 minutes. APCIMS: m/z 408.6 (M+1).

### Example 36 N-2-Adamantyl-2-(3,4-dihydroisoquinolin-2(1H)-yl)acetamide

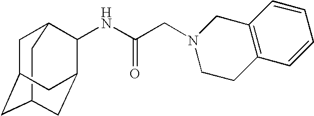

The titled compound was prepared analogously to Example 19 using instead N-2-adamantyl-2-chloroacetamide (150 milligrams) and 1,2,3,4-tetrahydroisoquinoline (175.6 milligrams), to provide the titled compound as a TFA salt (131.2 milligrams) with retention time of 13.6 minutes. APCIMS: m/z 325.5 (M+1).

### Example 37 2-(4-hydroxypiperidin-1-yl)-N-[(5R,7S)-3-phenyl-1-adamantyl]acetamide

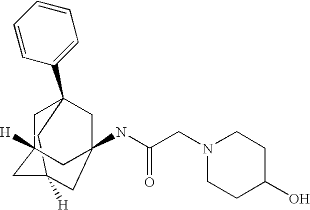

The titled compound was prepared analogously to Example 19 using instead 2-chloro-N-(3-phenyl-1-adamantyl)acetamide (157 milligrams) and 4-hydroxypiperidine (50.3 milligrams) to provide the titled compound as a TFA salt (10.6 milligrams) with retention time of 13.6 minutes APCIMS: m/z 367.5 (M+1). ¹H NMR (400 MHz, MeOD) δ ppm 1.46-1.57 (2H, m) 1.71 (2H, m) 1.78-1.89 (6H, m) 1.96-2.06 (4H, m) 2.11 (2H, s) 2.18-2.27 (4H, m) 2.67-2.77 (2H, m) 2.85 (2H, s) 3.26 (2H, dt, J=3.28, 1.64 Hz) 3.51-3.61 (1H, m, J=8.91, 8.91, 4.42, 4.04 Hz) 7.06-7.13 (1H, m) 7.21-7.27 (2H, m) 7.28-7.34 (2H, m).

### Example 38 N¹-2-Adamantyl-N²-benzyl-N²-methylglycinamide

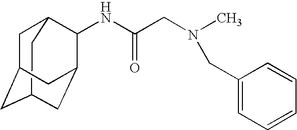

The titled compound was prepared analogously to Example 19 using instead N-2-adamantyl-2-chloroacetamide (102.8 milligrams) and N-benzylmethylamine (88 milligrams), to provide the titled compound as a TFA salt (42.2 milligrams) with retention time of 13.7 minutes APCIMS: m/z 313.5 (M+1).

### Example 39 N-2-Adamantyl-2-{4-[2-(trifluoromethyl)phenoxy]piperidin-1-yl}acetamide

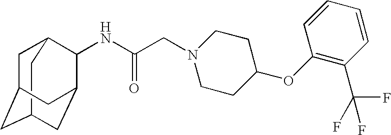

The titled compound was prepared analogously to Example 19 using instead N-2-adamantyl-2-chloroacetamide (104 milligrams), 4-(2-trifluoromethyl0phenoxy)piperidine.HCl (142 milligrams), K₂CO₃ (315 milligrams) and DMSO (2 milliliter) as a TFA salt (107.6 milligrams) with retention time of 16.5 minutes APCIMS: m/z 437.5 (M+1).

### Example 40 N-[(5R,7S)-3-(4-Fluorophenyl)-1-adamantyl]-2-(4-hydroxypiperidin-1-yl)acetamide

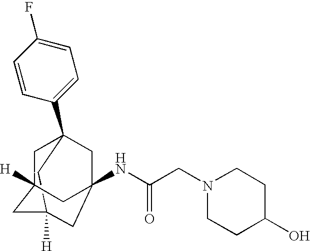

The titled compound was prepared analogously to Example 19 using instead 2-chloro-N-[3-(4-fluorophenyl)-1-adamantyl]acetamide (100 milligrams) and 4-hydroxypiperidine (95 milligrams) to provide the titled compound as a TFA salt and weighed 40 milligrams with retention time of 13.9 minutes APCIMS: m/z 387.5 (M+1).

### Preparation 40a [3-(4-Fluorophenyl)-1-adamantyl]amine

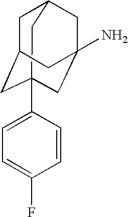

The titled compound was prepared analogously to Example 35c using instead

### Preparation 40b 2-Chloro-N-[3-(4-fluorophenyl)-1-adamantyl]acetamide

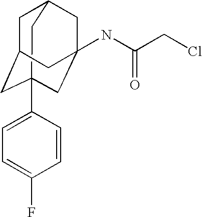

The titled compound was prepared analogously to Preparation 14a using instead chloroacetyl chloride (0.25 milliliter), anhydrous methylene chloride (20 milliliter), [3-(4-fluorophenyl)-1-adamantyl]amine (684 milligrams), K₂CO₃ (1150 milligrams) and weighed 600 milligrams. APCIMS: m/z 322.1 (M+1).

### Example 41 Methyl 1-(2-{[(5R,7S)-3-(4-fluorophenyl)-1-adamantyl]amino}-2-oxoethyl)piperidine-4-carboxylate

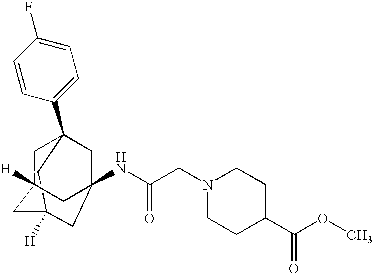

The titled compound was prepared analogously to Example 19 using instead 2-chloro-N-[3-(4-fluorophenyl)-1-adamantyl]acetamide (100 milligrams) and methyl isonipecotate (133.5 milligrams) to provide the titled compound as a TFA salt and weighed 4.5 milligrams with retention time of 15.24 minutes APCIMS: m/z 429.5 (M+1).

### Example 42 N-[(5R,7S)-3-(4-Fluorophenyl)-1-adamantyl]-2-piperidin-1-ylacetamide

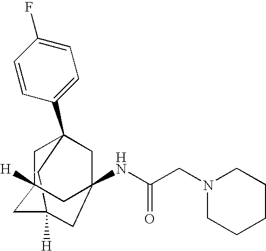

The titled compound was prepared analogously to Example 19 using instead 2-chloro-N-[3-(4-fluorophenyl)-1-adamantyl]acetamide (100 milligrams) and piperidine (66.8 milligrams) to provide the titled compound as a TFA salt and weighed 34.5 milligrams with retention time of 15.2 minutes APCIMS: m/z 371.5 (M+1).

### Example 43 N-[(5R,7S)-3-(4-Fluorophenyl)-1-adamantyl]-2-(3-methyl-5,6-dihydro[1,2,4]triazolo[4,3-a]pyrazin-7(8H)-yl)acetamide

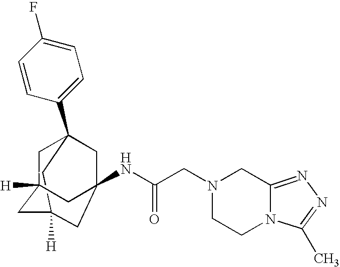

The titled compound was prepared analogously to Example 19 using instead 2-chloro-N-[3-(4-fluorophenyl)-1-adamantyl]acetamide (100 milligrams) and 3-methyl-5,6,7,8-tetrahydro[1,2,4]triazolo[4,3-a]pyrazine (90 milligrams) to provide the titled compound as a TFA salt and weighed 89.7 milligrams with retention time of 14.5 minutes APCIMS: m/z 424.5 (M+1).

### Example 44 2-(3,4-tetrahydroisoquinolin-2(1H)-yl)-N-[5R,7S)-3-(4-fluorophenyl)-1-adamantyl]acetamide

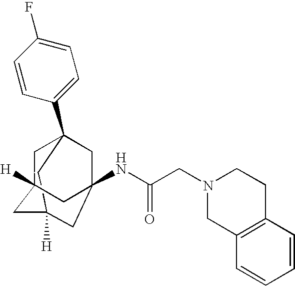

The titled compound was prepared analogously to Example 19 using instead 2-chloro-N-[3-(4-fluorophenyl)-1-adamantyl]acetamide (100 milligrams) and 1,2,3,4-tetrahydroisoquinoline (130 milligrams) to provide the titled compound as a TFA salt and weighed 17.8 milligrams with retention time of 16.8 minutes APCIMS: m/z 419.5 (M+1).

### Example 45 N-[(5R,7S)-3-(4-fluorophenyl)-1-adamantyl]-2-(4-pyridin-2-ylpiperazin-1-yl)acetamide

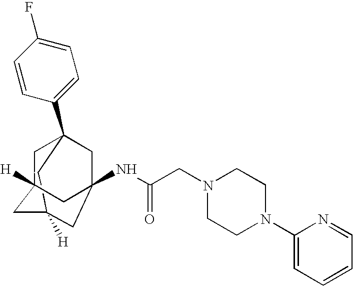

The titled compound was prepared analogously to Example 19 using instead 2-chloro-N-[3-(4-fluorophenyl)-1-adamantyl]acetamide (100 milligrams) and 1-2pyridyl-piperazine (75 milligrams) to provide the titled compound as a TFA salt and weighed 17.5 milligrams with retention time of 13.1 minutes APCIMS: m/z 449.2 (M+1).

### Example 46 N-2-Adamantyl-2-[4-(pyridin-2-ylmethyl)piperazin-1-yl]acetamide

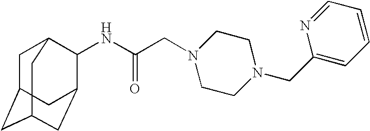

N-2-adamantyl-2-chloroacetamide (152 milligrams), 2-pyridylmethyl piperazine (128 milligrams), K₂CO₃ (454 milligrams) and DMSO (2 milliliter) were combined. After stirring at 40° C. for 24 hours, the reaction mixture was stopped, cooled to R.T., filtered to give a residue that precipitated at R.T to give the desired product as a white solid (68.9 milligrams). APCIMS: m/z 358.3 (M+1).

### Method E

### Example 47 N-1-Adamantyl-2-[4-(2-aminoethyl)piperazin-1-yl]acetamide

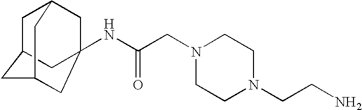

N-1-adamantyl-2-chloroacetamide (250 milligrams) and N-(2-aminoethyl)piperazine (147 milligrams), K₂CO₃ (300 milligrams) and DMSO (3 milliliter) were combined at room temperature The reaction flask was capped with yellow stopper. After stirring at 40° C. for 20 hours, the reaction mixture was stopped, cooled to room temperature, diluted with methanol (1 milliliters), and filtered. The solvent was removed under reduced pressure and the residue was purified using reversed phase Kromasil C18, 0.05% TFA in water and acetonitrile to provide the titled product as a TFA salt (182.3 milligrams) with retention time of 9.3 minutes. APCIMS: m/z 321.3 (M+1). Kᵢ:NT.

The titled compound was prepared analogously to Example 47 using instead N-1-adamantyl-2-chloroacetamide (250 milligrams) and 1-ethoxycarbonylpiperazine (190 milligrams) providing the titled compound, using MP-CO3 as the free base (145 milligrams) with retention time of 12.1 minutes. APCIMS: m/z 350.2 (M+1).

### Example 48 Ethyl 4-[2-(1-adamantylamino)-2-oxoethyl]piperazine-1-carboxylate

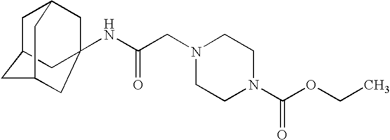

### Example 49N-1-Adamantyl-2-piperidin-1-ylacetamide

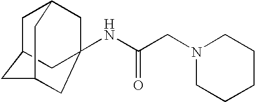

The titled compound was prepared analogously to Example 47 using instead N-1-adamantyl-2-chloroacetamide (250 milligrams) and piperidine (96 milligrams), K₂CO₃ (300 milligrams) to provide the titled compound as a TFA salt (77.7 milligrams) with retention time of 11.7 minutes. APCIMS: m/z 277.2 (M+1).

### Example 50 N-1-Adamantyl-2-[4-(2-oxo-2-pyrrolidin-1-ylethyl)piperazin-1-yl]acetamide

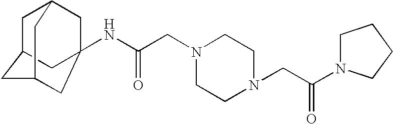

The titled compound was prepared analogously to Example 47 using instead N-1-adamantyl-2-chloroacetamide (250 milligrams) and N-(2-(1-piperazino)acetyl-pyrrolidine (217 milligrams) to provide the titled compound as a TFA salt (176 milligrams) with retention time of 11.1 minutes. APCIMS: m/z 389.3 (M+1).

### Example 51 N-1-Adamantyl-2-(1,3-dihydro-2H-isoindol-2-yl)acetamide

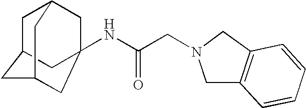

The titled compound was prepared analogously to Example 47 using instead N-1-adamantyl-2-chloroacetamide (250 milligrams) and isoindoline (131 milligrams) to provide the titled compound as a TFA salt (210.7 milligrams) with retention time of 13.1 minutes. APCIMS: m/z 311.2 (M+1). ¹H NMR (400 MHz, MeOD) δ ppm 1.19-1.30 (1H, m) 1.68 (6H, s) 2.00 (8H, s) 3.25-3.27 (3H, m) 3.97 (4H, s) 7.13-7.19 (4H, m).

### Example 52 N-1-Adamantyl-2-(4-benzylpiperazin-1-yl)acetamide

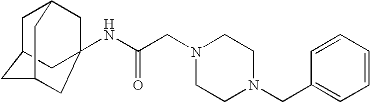

The titled compound was prepared analogously to Example 47 using instead N-1-adamantyl-2-chloroacetamide (250 milligrams) and 1-benzylpiperazine (207 milligrams) to provide the titled compound weighed (32.8 milligrams) with retention time of 12.9 minutes. APCIMS: m/z 368.3 (M+1).

### Example 53 N-1-Adamantyl-2-(2,6-dimethylmorpholin-4-yl)acetamide

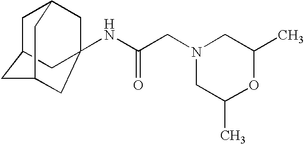

The titled compound was prepared analogously to Example 47 using instead N-1-adamantyl-2-chloroacetamide (250 milligrams) and 2,6-dimethylmorpholine (131 milligrams) to provide the titled compound as a TFA salt (172 milligrams) with retention time of 11.9 minutes. APCIMS: m/z 307.2 (M+1).

### Example 54 N-1-Adamantyl-2-(3,4-dihydroisoquinolin-2(1H)-yl)acetamide

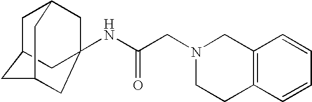

The titled compound was prepared analogously to Example 47 using instead N-1-adamantyl-2-chloroacetamide (250 milligrams) and 1,2,3,4-tetrahydroisoquinoline (147 milligrams), K₂CO₃ (300 milligrams) and DMSO (2 milliliter) weighed (121 milligrams) after free base with MP-CO₃. The TFA salt had retention time of 13.5 minutes. APCIMS: m/z 325.2 (M+1). ¹H NMR (400 MHz, MeOD) δ ppm 1.62-1.71 (6H, m) 1.95-2.04 (10H, m) 2.75 (2H, t, J=5.94 Hz) 2.87 (2H, t, J=5.81 Hz) 3.03 (2H, s) 3.65 (2H, s) 6.95-7.00 (1H, m) 7.03-7.10 (3H, m).

### Example 55 N-1-Adamantyl-2-(4-hydroxypiperidin-1-yl)acetamide

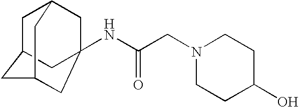

The titled compound was prepared analogously to Example 47 using instead N-1-adamantyl-2-chloroacetamide (251 milligrams) and 4-hydroxypiperidine (217 milligrams), to provide the titled compound as a white solid after free base with MP-CO₃. The TFA salt had a retention time of 10.6 minutes. APCIMS: m/z 293.2 (M+1).

### Example 56 N¹-1-Adamantyl-N²-[2-(dimethylamino)ethyl]glycinamide

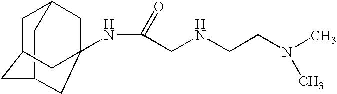

The titled compound was prepared analogously to Example 47 using instead N-1-adamantyl-2-chloroacetamide (260 milligrams) and N,N-dimethylethylenediamine (96 milligrams) the titled compound was provided as a TFA salt (85.4 milligrams) with a retention time of 9.1 minutes. APCIMS: m/z 280.4 (M+1).

### Example 57 N¹-1-Adamantyl-N²-cyclohexyl-N²-methylglycinamide

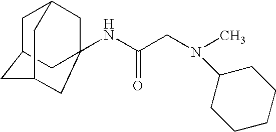

The titled compound was prepared analogously to Example 47 using instead N-1-adamantyl-2-chloroacetamide (251.6 milligrams) and N-methylcyclohexylamine (136.3 milligrams), K₂CO₃ (305.2 milligrams) to provide the titled product as a TFA salt (209.5 milligrams) with retention time of 13.7 minutes. APCIMS: m/z 305.5 (M+1). ¹H NMR (400 MHz, MeOD) δ ppm 1.15-1.26 (1H, m) 1.37-1.47 (4H, m) 1.68-1.76 (7H, m) 1.88-1.99 (3H, s) 1.99-2.09 (1H, m) 2.81 (3H, s) 3.20 (1H, tt, J=11.75, 3.41 Hz) 3.62 (1H, d, J=15.41 Hz) 3.92 (1H, d, J=15.66 Hz).

### Example 58 1-[2-(1-Adamantylamino)-2-oxoethyl]piperidine-4-carboxamide

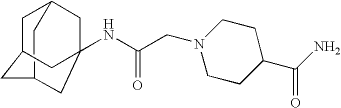

The titled compound was prepared analogously to Example 47 using instead N-1-adamantyl-2-chloroacetamide (125 milligrams) and isonipecotamide (71 milligrams), K₂CO₃ (152 milligrams) and DMSO (2 milliliter) weighed 61.1 milligrams after free base with MP-CO₃. The TFA salt had a retention time of 10.4 minutes. APCIMS: m/z 320.2 (M+1). ¹H NMR (400 MHz, MeOD) δ ppm 1.70-1.82 (12H, m) 1.99-2.09 (10H, m) 2.15-2.26 (3H, m) 2.84-2.93 (4H, m).

### Example 59 N-2-Adamantyl-2-(4-pyridin-2-ylpiperazin-1-yl)acetamide

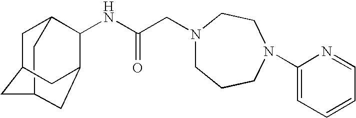

The titled compound was prepared analogously to Example 47 using instead N-2-adamantyl-2-chloroacetamide (101 milligrams) and 1-(2-pyridyl)-piperazine (100 milligrams), to provide the titled compound as a TFA salt (126.1 milligrams) with retention time of 10 minutes. APCIMS: m/z 355.3 (M+1). ¹H NMR (400 MHz, MeOD) δ ppm 1.42 (2H, d, J=12.38 Hz) 1.57-1.77 (15H, m) 3.07 (1H, dt, J=3.28, 1.64 Hz) 3.71-3.85 (8H, m) 6.80 (1H, t, J=6.57 Hz) 7.07 (1H, d, J=9.09 Hz) 7.75 (1H, ddd, J=8.97, 7.20, 1.77 Hz) 7.89 (1H, dd, J=5.81, 1.01 Hz).

### Method F

### Example 60 1-(2-oxo-2-{[(5R,7S)-3-phenyl-1-adamantyl]amino}ethyl)piperidine-4-carboxamide

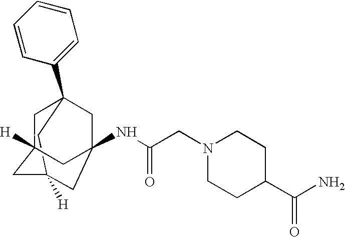

The titled compound was prepared analogously to Preparation 14a using instead 2-C-chloro-N-(3-phenyl-1-adamantyl)acetamide (136 milligrams), 4-piperidine carboxamide (58 milligrams), K₂CO₃ (191 milligrams) and DMSO (3 milliliter) as a TFA salt (22.7 milligrams) with retention time of 13.3 minutes APCIMS: m/z 396.5 (M+1).

### Example 61 Benzyl [3-(4-fluorophenyl)-1-adamantyl]carbamate

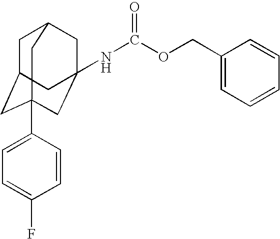

The titled compound was prepared analogously to Preparation 29a.

The structures, physical data, biological data, and associative Method are further described below in Table 1.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

TABLE 1

| | | Structure | | APCl-MS |
|---|---|---|---|---|
| Example | Kᵢ | IUPAC Name | Method | m/z |
| 2 | NT | 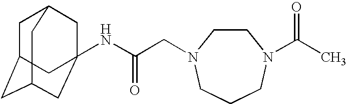 | A | 334.3 |
| | | 2-(4-Acetyl-1,4-diazepan-1-yl)- | | |
| | | N-1-adamantylacetamide | | |
| 3 | NT | 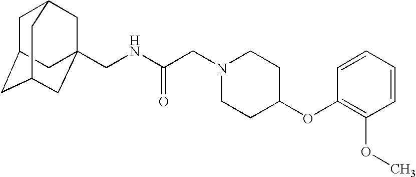 | A | 413.3 |
| | | N-(1-Adamantylmethyl)-2-[4-(2- | | |
| | | methoxyphenoxy)piperidin-1- | | |
| | | yl]acetamide | | |
| 4 | 6.5 | 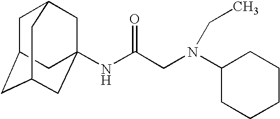 | A | 319.3 |
| | | N¹-1-Adamantyl-N²-cyclohexyl- | | |
| | | N²-ethylglycinamide | | |
| 5 | NT | 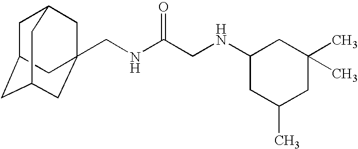 | A | 347.3 |
| | | N¹-(1-Adamantylmethyl)-N²- | | |
| | | (3,3,5-trimethylcyclohexyl)glycinamide | | |
| 6 | NT | 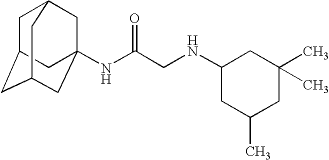 N¹-1-Adamantyl-N²-(3,3,5-trimethylcyclohexyl)glycinamide | A | 333.3 3 |
| 7 | 28 | 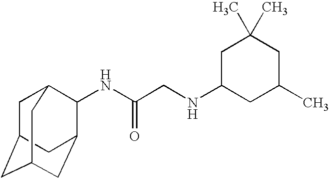 | A | 333.3 |
| | | N¹-2-Adamantyl-N²- | | |
| | | (3,3,5-trimethylcyclohexyl)glycinamide | | |
| 8 | 15 | 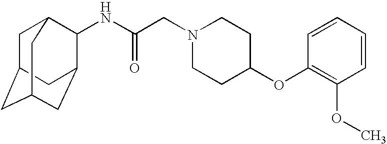 | A | 399.3 |
| | | N-2-Adamantyl-2-[4-(2- | | |
| | | methoxyphenoxy)piperidin-1- | | |
| | | yl]acetamide | | |
| 9 | 1 | 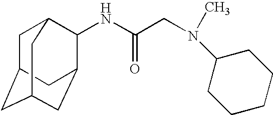 | A | 305.5 |
| | | N¹-2-Adamantyl-N²-cyclohexyl- | | |
| | | N²-methylglycinamide | | |
| 10 | 3 | 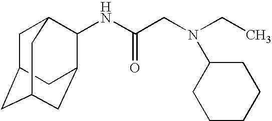 | A | 319.5 |
| | | N¹-2-Adamantyl-N²-cyclohexyl- | | |
| | | N²-ethylglycinamide | | |
| 11 | 3 | 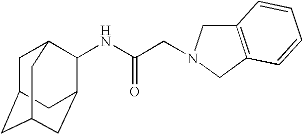 | A | 311.5 |
| | | N-2-Adamantyl-2-(1,3-dihydro- | | |
| | | 2H-isoindol-2-yl)acetamide | | |
| 14 | NT | 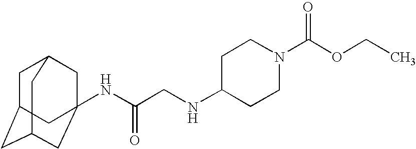 | C | 364.3 |
| | | Ethyl 4-{[2-(1-adamantylamino)- | | |
| | | 2-oxoethyl]amino}piperidine-1- | | |
| | | carboxylate | | |
| 15 | NT | 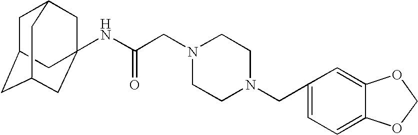 | C | 412.3 |
| | | N-1-Adamantyl-2-[4-(1,3- | | |
| | | benzodioxol-5-ylmethyl)piperazin-1- | | |
| | | yl]acetamide | | |
| 16 | NT | 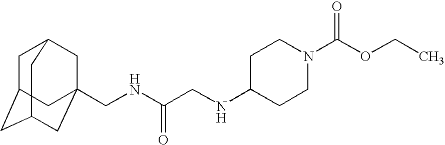 | C | 378.3 |
| | | Ethyl 4-({2-[(1- | | |
| | | adamantylmethyl)amino]-2- | | |
| | | oxoethyl}amino)piperidine-1-carboxylate | | |
| 17 | NT | 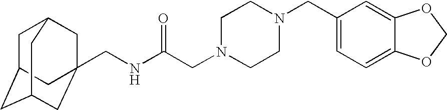 | C | 426.3 |
| | | N-(1-Adamantylmethyl)-2-[4- | | |
| | | (1,3-benzodioxol-5-ylmethyl)piperazin-1- | | |
| | | yl]acetamide | | |
| 18 | NT | 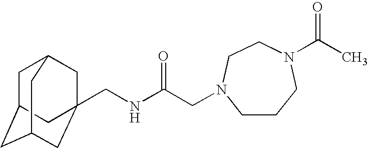 | C | 348.3 |
| | | 2-(4-Acetyl-1,4-diazepan-1-yl)- | | |
| | | N-(1-adamantylmethyl)acetamide | | |
| 19 | NT | 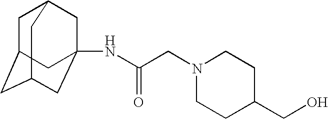 | D | 307.3 |
| | | N-(1-Adamantyl)-2-[4- | | |
| | | (hydroxymethyl)piperidin-1-yl]acetamide | | |
| 20 | NT | 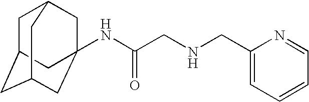 | D | 300.2 |
| | | N¹-1-Adamantyl-N²-(pyridin-2- | | |
| | | ylmethyl)glycinamide | | |
| 21 | NT | 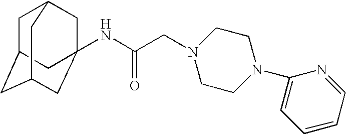 | D | 355.3 |
| | | N-1-Adamantyl-2-(4-pyridin-2- | | |
| | | ylpiperazin-1-yl)acetamide | | |
| 22 | 4 | 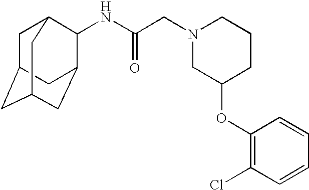 | D | 403.2 |
| | | N-2-Adamantyl-2-[3-(2- | | |
| | | chlorophenoxy)piperidin-1-yl]acetamide | | |
| 23 | NT | 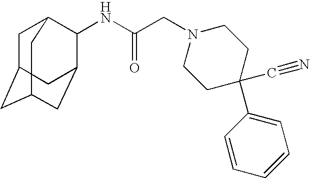 | D | 378.5 |
| | | N-2-Adamantyl-2-(4-cyano-4- | | |
| | | phenylpiperidin-1-yl)acetamide | | |
| 24 | 3 | 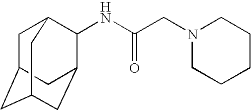 | D | 277.4 |
| | | N-2-Adamantyl-2-piperidin-1- | | |
| | | ylacetamide | | |
| 25 | 3 | 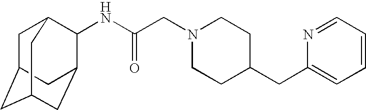 | D | 368.5 |
| | | N-2-Adamantyl-2-[4-(pyridin-2- | | |
| | | ylmethyl)piperidin-1-yl]acetamide | | |
| 26 | 7.5 | 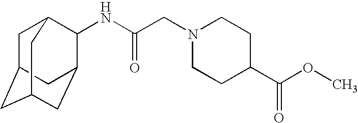 | D | 335.3 |
| | | Methyl 1-[2-(2-adamantylamino)- | | |
| | | 2-oxoethyl]piperidine-4-carboxylate | | |
| 27 | NT | 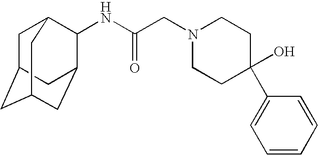 | D | 369.5 |
| | | N-2-Adamantyl-2-(4-hydroxy-4- | | |
| | | phenylpiperidin-1-yl)acetamide | | |
| 28 | 10 | 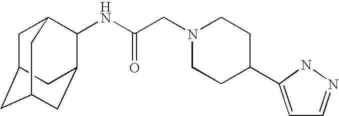 | D | 343.3 |
| | | N-2-Adamantyl-2-[4-(1H- | | |
| | | pyrazol-5-yl)piperidin-1-yl]acetamide | | |
| 29 | NT | 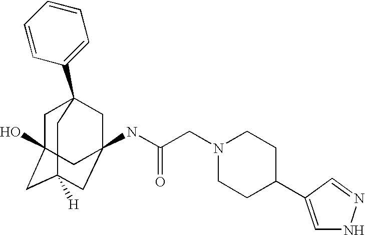 | D | 419.6 |
| | | N-[(5R,7S)-3-phenyl-1- | | |
| | | adamantyl]-2-[4-(1H-pyrazol-4- | | |
| | | yl)piperidin-1-yl]acetamide | | |
| 30 | NT | 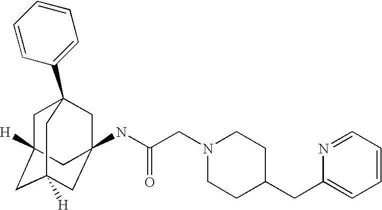 | D | 444.3 |
| | | N-[(5R,7S)-3-phenyl-1- | | |
| | | adamantyl]-2-[4-(pyridin-2- | | |
| | | ylmethyl)piperidin-1-yl]acetamide | | |
| 31 | NT | 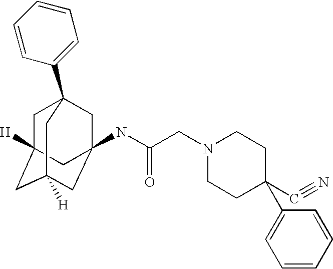 | D | 454.6 |
| | | 2-(4-cyano-4-phenylpiperidin-1- | | |
| | | yl)-N-[(5R,7S)-3-phenyl-1- | | |
| | | adamantyl]acetamide | | |
| 32 | NT | 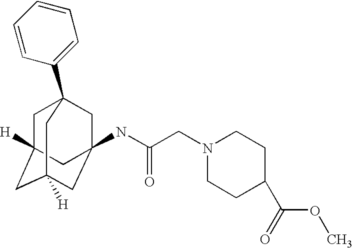 | D | 411.6 |
| | | Methyl1-(2-oxo-2-{[(5R,7S)-3- | | |
| | | phenyl-1- | | |
| | | adamantyl]amino}ethyl)piperidine-4- | | |
| | | carboxylate | | |
| 33 | NT | 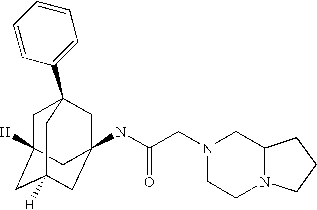 | D | 419.6 |
| | | 2-(Hexahydropyrrolo[1,2- | | |
| | | a]pyrazin-2(1H)-yl)-N-[(5R,7S)-3-phenyl- | | |
| | | 1-adamanty]acetamide | | |
| 34 | NT | 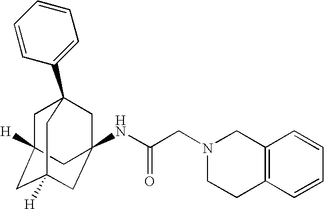 | D | 401.6 |
| | | 2-(3,4-dihydroisoquinolin-2(1H)- | | |
| | | yl)-N-[(5R,7S)-3-phenyl-1- | | |
| | | adamantyl]acetamide | | |
| 35 | NT | 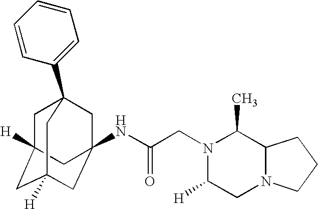 | D | 408.6 |
| | | 2-[(3S,8aS)-3- | | |
| | | Methylhexahydropyrrolo[1,2-a]pyrazin- | | |
| | | 2(1H)-yl]-N-[(5R,7S)-3-phenyl-1- | | |
| | | adamantyl]acetamide | | |
| 36 | 2 | 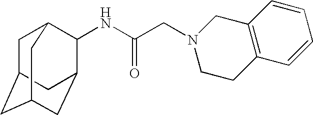 | D | 325.5 |
| | | N-2-Adamantyl-2-(3,4- | | |
| | | dihydroisoquinolin-2(1H)-yl)acetamide | | |
| 37 | NT | 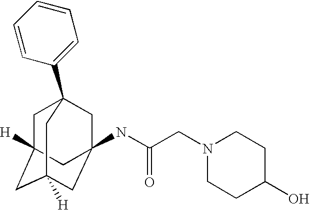 | D | 367.5 |
| | | 2-(4-hydroxypiperidin-1-yl)-N- | | |
| | | [(5R,7S)-3-phenyl-1- | | |
| | | adamantyl]acetamide | | |
| 38 | 4 | 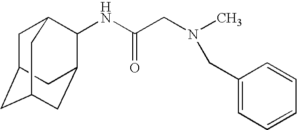 | D | 313.5 |
| | | 1-2-Adamantyl-N²-benzyl-N²- | | |
| | | methylglycinamide | | |
| 39 | 14 | 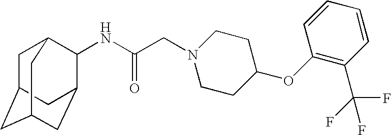 | D | 437.5 |
| | | N-2-Adamantyl-2-{4-[2- | | |
| | | (trifluoromethyl)phenoxy]piperidin-1- | | |
| | | yl}acetamide | | |
| 40 | NT | 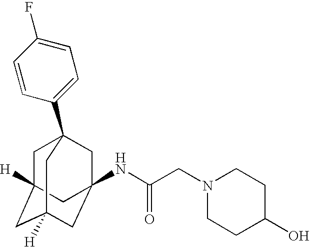 | D | 387.5 |
| | | N-[(5R,7S)-3-(4-Fluorophenyl)- | | |
| | | 1-adamantyl]-2-(4-hydroxypiperidin-1- | | |
| | | yl)acetamide | | |
| 41 | NT | 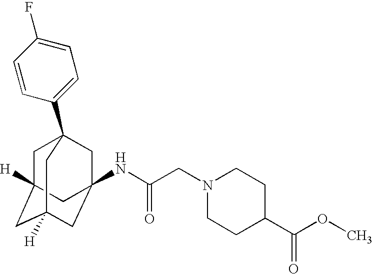 | D | 429.5 |
| | | Methyl 1-(2-{[(5R,7S)-3-(4- | | |
| | | fluorophenyl)-1-adamantyl]amino}-2- | | |
| | | oxoethyl)piperidine-4-carboxylate | | |
| 42 | NT | 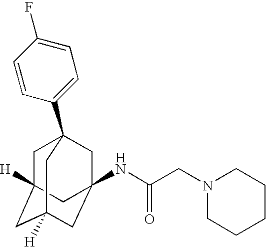 | D | 371.5 |
| | | N-[(5R,7S)-3-(4-Fluorophenyl)- | | |
| | | 1-adamantyl]-2-piperidin-1-ylacetamide | | |
| 43 | NT | 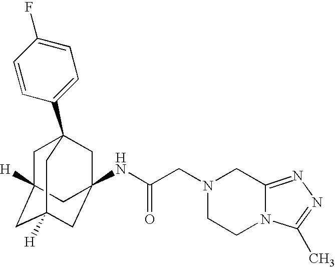 | D | 424.5 |
| | | N-[5R,7S)-3-(4-Fluorophenyl)-1- | | |
| | | adamantyl]-2-(3-methyl-5,6- | | |
| | | dihydro[1,2,4]triazolo[4,3-a]pyrazin- | | |
| | | 7(8H)-yl)acetamide | | |
| 44 | NT | 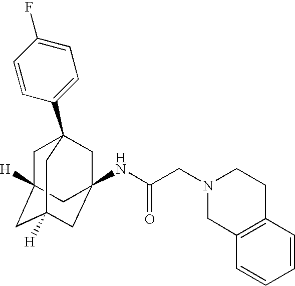 | D | 419.5 |
| | | 2-(3,4-tetrahydroisoquinolin- | | |
| | | 2(1H)-yl)-N-[5R,7S)-3-(4-fluorophenyl)- | | |
| | | 1-adamantyl]acetamide | | |
| 45 | NT | 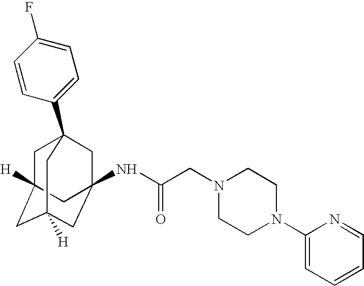 | D | 449.2 |
| | | N-[(5R,7S)-3-(4-fluorophenyl)-1- | | |
| | | adamantyl]-2-(4-pyridin-2-ylpiperazin-1- | | |
| | | yl)acetamide | | |
| 46 | NT | 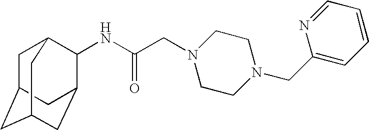 | D | 378.5 |
| | | N-2-Adamantyl-2-[4-(pyridin-2- | | |
| | | ylmethyl)piperazin-1-yl]acetamide | | |
| 48 | NT | 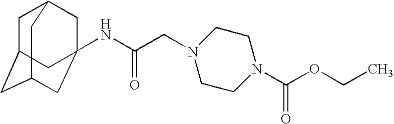 | E | 350.2 |
| | | Ethyl 4-[2-(1-adamantylamino)- | | |
| | | 2-oxoethyl]piperazine-1-carboxylate | | |
| 49 | NT | 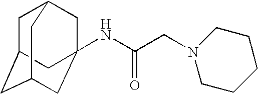 | E | 277.2 |
| | | N-1-Adamantyl-2-piperidin-1- | | |
| | | ylacetamide | | |
| 50 | NT | 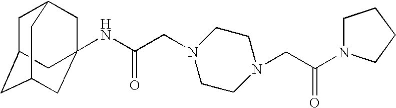 | E | 389.3 |
| | | N-1-Adamantyl-2-[4-(2-oxo-2- | | |
| | | pyrrolidin-1-ylethyl)piperazin-1- | | |
| | | yl]acetamide | | |
| 51 | 52 | 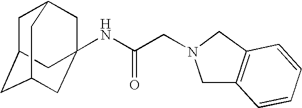 | E | 311.2 |
| | | N-1-Adamantyl-2-(1,3-dihydro- | | |
| | | 2H-isoindol-2-yl)acetamide | | |
| 52 | NT | 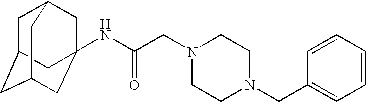 | E | 368.3 |
| | | N-1-Adamantyl-2-(4- | | |
| | | benzylpiperazin-1-yl)acetamide | | |
| 53 | NT | 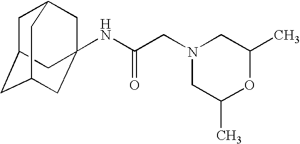 | E | 307.2 |
| | | N-1-Adamantyl-2-(2,6- | | |
| | | dimethylmorpholin-4-yl)acetamide | | |
| 54 | 4 | 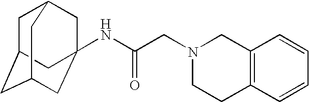 | E | 325.2 |
| | | N-1-Adamantyl-2-(3,4- | | |
| | | dihydroisoquinolin-2(1H)-yl)acetamide | | |
| 55 | NT | 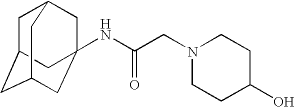 | E | 293.2 |
| | | N-1-Adamantyl-2-(4- | | |
| | | hydroxypiperidin-1-yl)acetamide | | |
| 56 | NT | 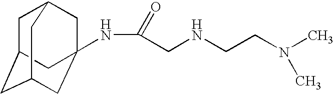 | E | 280.4 |
| | | N¹-1-Adamantyl-N²-[2- | | |
| | | (dimethylamino)ethyl]glycinamide | | |
| 57 | 3 | 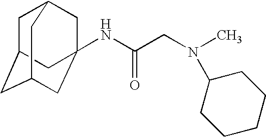 | E | 319.5 |
| | | N¹1-Adamantyl-N²-cyclohexyl- | | |
| | | N²-methylglycinamide | | |
| 58 | NT | 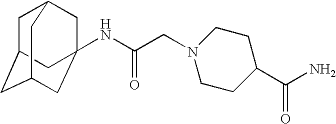 | E | 320.2 |
| | | 1-[2-(1-Adamantylamino)-2- | | |
| | | oxoethyl]piperidine-4-carboxamide | | |
| 59 | 3 | 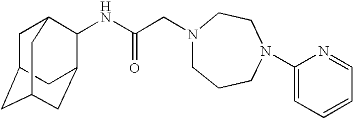 | E | 355.3 |
| | | N-2-Adamantyl-2-(4-pyridin-2- | | |
| | | ylpiperazin-1-yl)acetamide | | |

We claim:

1. A compound of formula (I): 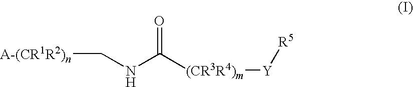
wherein:
each R¹, R², R³, and R⁴ is independently selected from H and (C₁-C₆)alkyl;
Y is selected from the group consisting of O, S, and NR⁶;
each R⁵ and R⁶ is independently selected from the group consisting of H, (C₁-C₆) alkyl, (C₂-C₆) alkenyl, (C₂-C₆) alkynyl, —(CR⁷R⁸)ₜ(C₃-C₁₀)cycloalkyl, —(CR⁷R⁸)ₜ(C₆-C₁₀)aryl, and —(CR⁷R⁸)ₜ(4-11)-membered heterocyclyl;
or, where Y is NR⁶, R⁵ and R⁶ may optionally be taken together with the nitrogen atom to which they are attached to form a (4-11)-membered heterocyclyl, and the (4-11)-membered heterocyclyl may optionally be substituted by 1 to 5 R⁹ groups;
each R⁷ and R⁸ is independently selected from the group consisting of H, (C₁-C₆) alkyl, (C₂-C₆) alkenyl, and (C₂-C₆) alkynyl;
A is adamantyl;
n and m are independently selected from the group consisting of 0, 1, 2, and 3;
k is 1 or 2;
j is selected from the group consisting of 0, 1, and 2;
t, u, p, q and v are each independently selected from 0, 1, 2, 3, 4, and 5;
any carbon atom of A, R¹, R², R³, R⁴, R⁵, R⁶, R⁷, and, R⁸ may be optionally substituted by 1 to 5 R⁹ groups;
any nitrogen atom of R⁵ or R⁶ wherein R⁵ or R⁶ is a (4-11)-membered heterocyclyl are each optionally substituted by 1 to 5 R⁹ groups;
each R⁹ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR¹⁰R¹¹)ₜ(C₃-C₁₀)cycloalkyl, —(CR¹²R¹³)ₜ(C₆-C₁₀)aryl, —O—R¹², (C═O)—R¹², —(C═O)—O—R¹², —O—(C═O)—R¹², —O—(R¹²)—O—(R¹³), —NR¹²(C═O)—R¹³, —(C═O)—NR¹²R¹³, —NR¹²R¹³, —NR¹²OR¹³, —S(O)ₖNR¹²R¹³, —S(O)ⱼ(C₁-C₆)alkyl, —O—SO₂—R¹⁴, —NR¹⁴—S(O)ₖ—R¹⁵, —(CR¹⁴R¹⁵)ᵥ(C₆-C₁₀) aryl, —(CR¹⁴R¹⁵)ᵥ(4-11)-membered heterocyclyl, —(CR¹⁴R¹⁵)_{q}(C═O)(CR¹⁴R¹⁵)ᵥ(C₆-C₁₀)aryl, —(CR¹⁴R¹⁵)_{q}(C═O)(CR¹⁴R¹⁵)ᵥ(4-11)-membered heterocyclyl, —(CR¹⁴R¹⁵)ᵥO(CR¹⁴R¹⁵)_{q}(C₆-C₁₀)aryl, —(CR¹⁴R¹⁵)ᵥO(CR¹⁴R¹⁵)_{q}(4-11)-membered heterocyclyl, —(CR¹⁴R¹⁵)_{q}S(O)ⱼ(CR¹⁴R¹⁵)ᵥ(C₆-C₁₀)aryl, and —(CR¹⁴R¹⁵)_{q}S(O)ⱼ(CR¹⁴R¹⁵)ᵥ(4-11)-membered heterocyclyl;
any 1 or 2 carbon atoms of any (4-11)-membered heterocyclyl of the foregoing R⁹ groups are optionally substituted with an oxo (═O);
any carbon atom of any (C₁-C₆)alkyl, any (C₆-C₁₀)aryl and any (4-11)-membered heterocyclyl of the foregoing R⁹ groups are optionally substituted with 1 to 5 substituents independently selected from the group consisting of halo, cyano, nitro, —CF₃, —CFH₂, —CF₂H, trifluoromethoxy, azido, —OR¹⁶, —(C═O)—R¹⁶, —(C═)—O—R¹⁶, —O—(C═O)—R¹⁶, —NR¹⁶(C═O)—R¹⁷, (C═O)—NR¹⁶R¹⁷, —NR¹⁶R¹⁷, —NR¹⁶OR¹⁷, —S(O)ₖNR¹²R¹³, S(O)ⱼ(C₁-C₆)alkyl, —O—SO₂—R¹⁴, —NR¹⁴—S(O)ₖ—R¹⁵, (C₁-C₆)alkyl, (C₂-C₆)alkenyl, (C₂-C₆)alkynyl, (CR¹⁷R¹⁸)ᵤ(C₁-C₁₀)aryl, and (CR¹⁷R¹⁸)ᵤ(4-11)-membered heterocyclyl;
each R¹⁰, R¹¹, R¹², R¹³, R¹⁴R¹⁵, R¹⁶, R¹⁷, and R¹⁸ group is independently selected from the group consisting of H, (C₁-C₆)alkyl, —(CR¹⁹R²⁰)ₜ(C₃-C₁₀)cycloalkyl, —(CR¹⁹R²⁰)ₚ(C₆-C₁₀)aryl, and —(CR¹⁹R²⁰)ₚ(4-11)-membered heterocyclyl;
any 1 or 2 carbon atoms of the (4-11)-membered heterocyclyl of said each R¹⁰, R¹¹, R¹², R¹³, R¹⁴, R¹⁵, R¹⁶, R¹⁷, and R¹⁸ group is optionally substituted with an oxo (═O);
any carbon atom of any (C₁-C₆)alkyl, any (C₈-C₁₀)aryl and any (4-11)-membered heterocyclyl of the foregoing R¹⁰, R¹¹, R¹², R¹³, R¹⁴, R¹⁵, R¹⁶, R¹⁷, and R¹⁸ groups are optionally substituted with 1 to 5 substituents independently selected from the group consisting of halo, cyano, nitro, —NR²¹R²², —CF₃, —CHF₂, —CH₂F, trifluoromethoxy, (C₁-C₆)alkyl, (C₂-C₆)alkenyl, (C₂-C₆)alkynyl, hydroxy, and (C₁-C₆) alkoxy;
each R¹⁹, R²⁰, R²¹, and R²² group is independently selected from the group consisting of H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from hydroxy, halo, (C₁-C₆)alkyl, (C₁-C₆)alkoxy, amino, —NH(C₁-C₆)(alkyl) and —N(C₁-C₆) (alkyl)(C₁-C₆) alkyl;
or a pharmaceutically acceptable salt or solvate thereof.

2. The compound according to claim 1, wherein Y is O.

3. The compound according to claim 1, wherein Y is NR⁶.

4. The compound according to claim 3, wherein R⁵ and R⁶ are taken together with the nitrogen to which they are attached to form a (4-11)-membered heterocyclyl.

5. The compound according to claim 4, wherein the (4-11)-membered heterocyclyl is selected from the group consisting of pyrrolidinyl, indolyl, isoquinolinyl, piperazinyl, and piperidinyl.

6. A compound selected from the group consisting of: 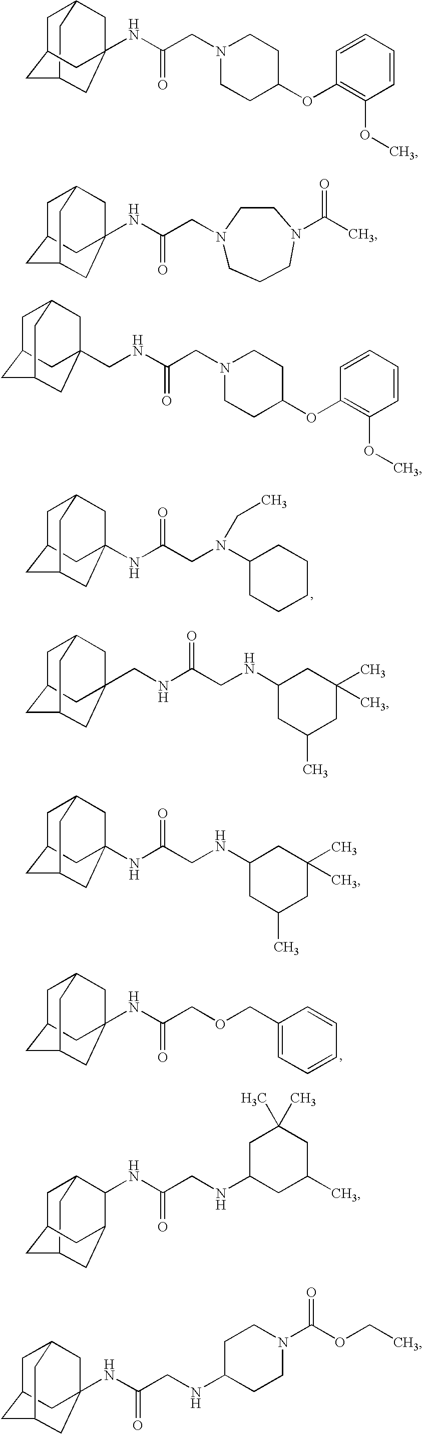 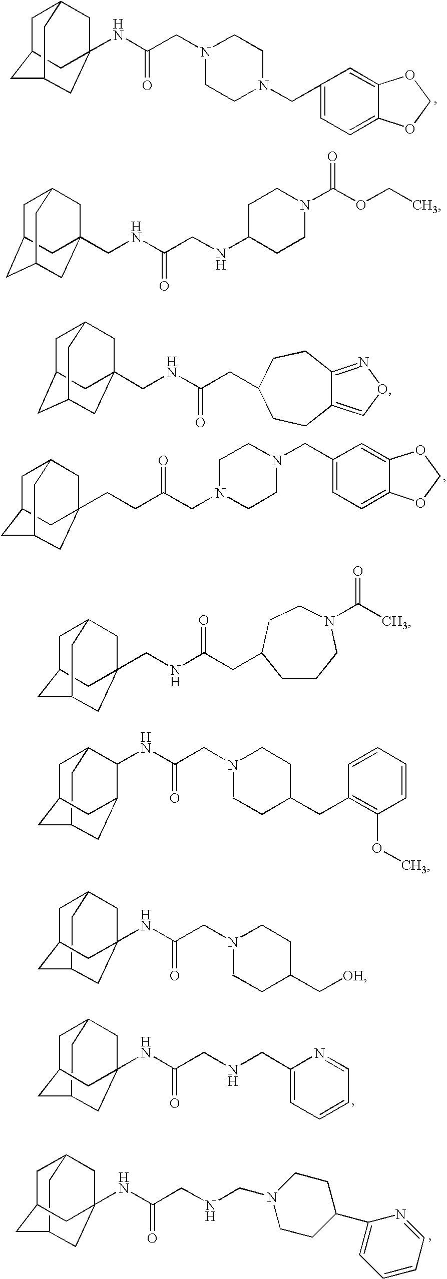 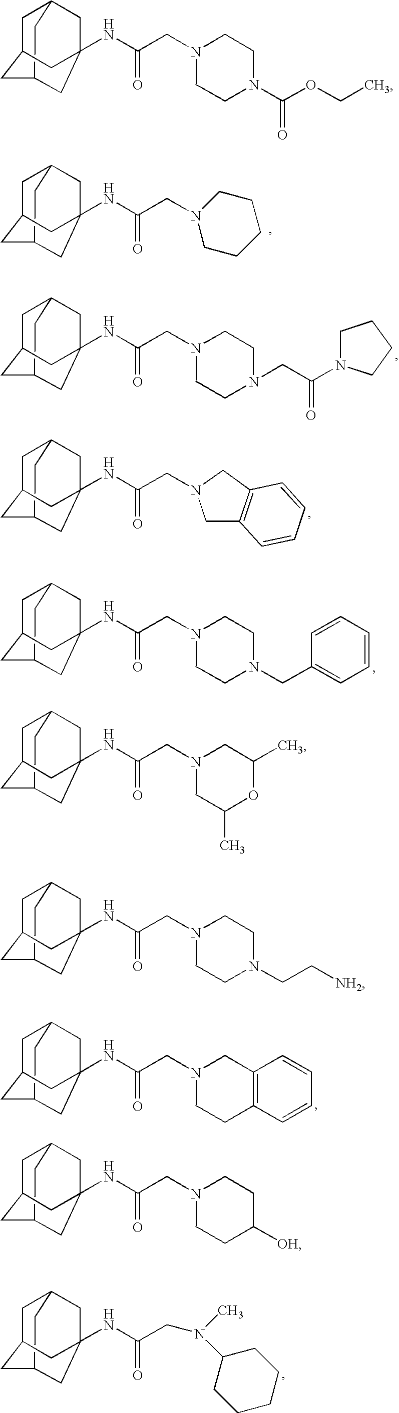 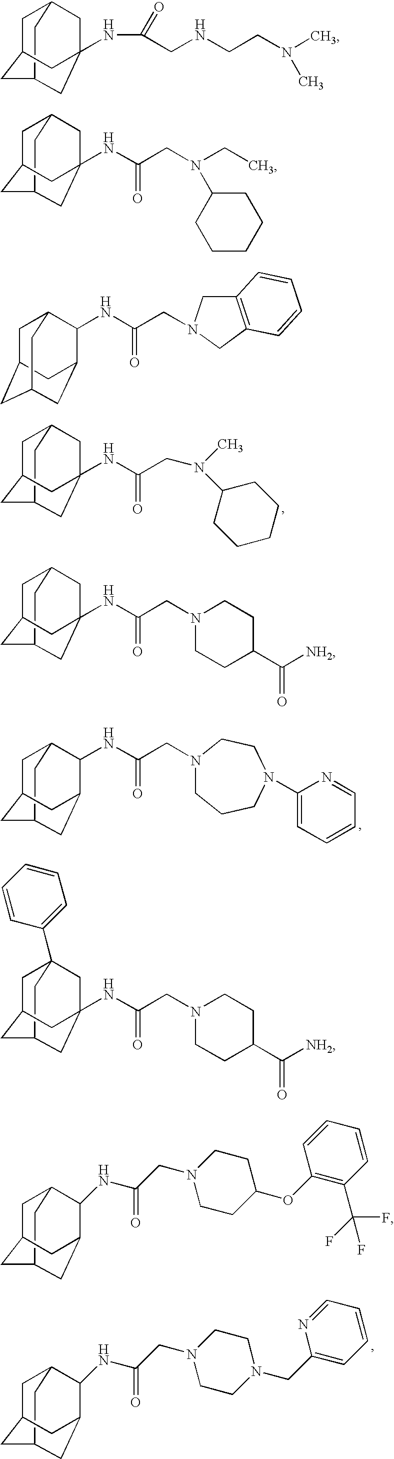 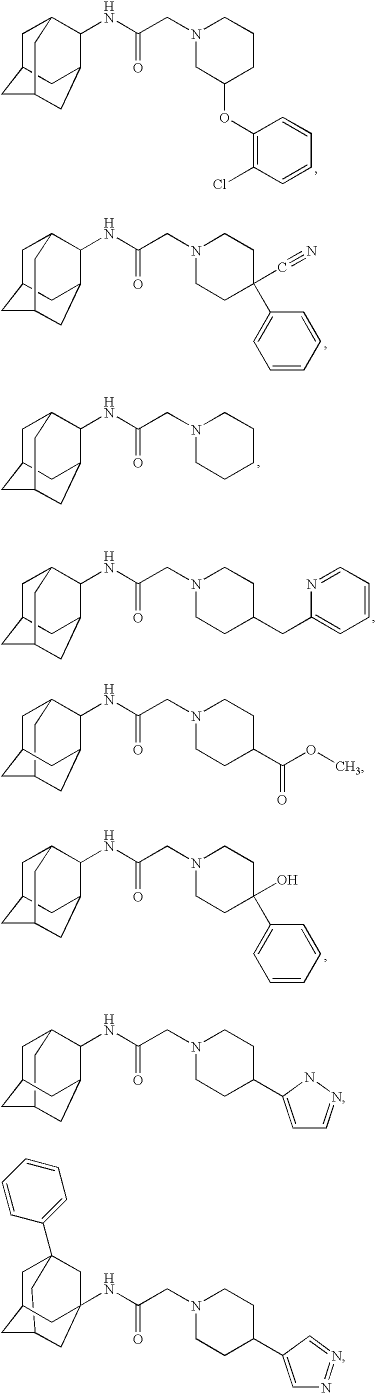 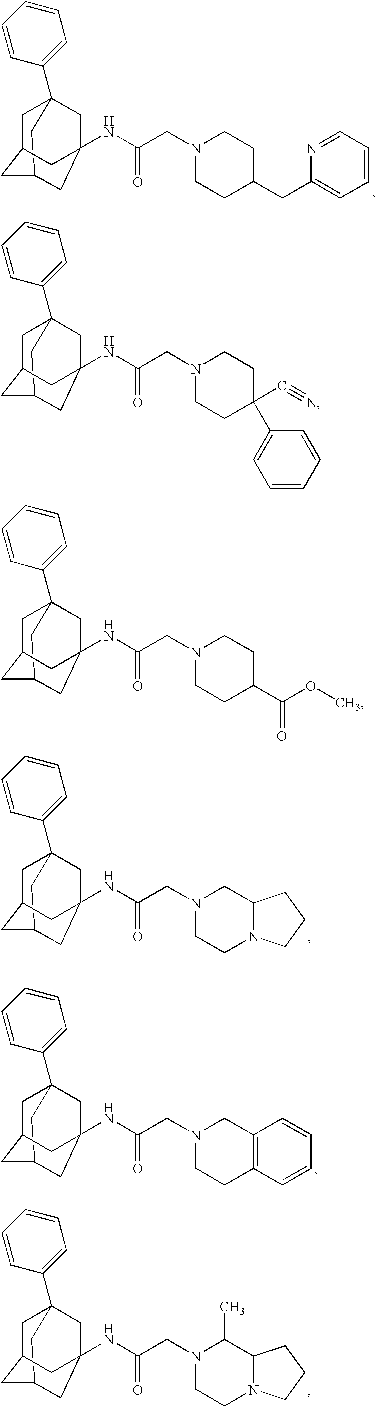 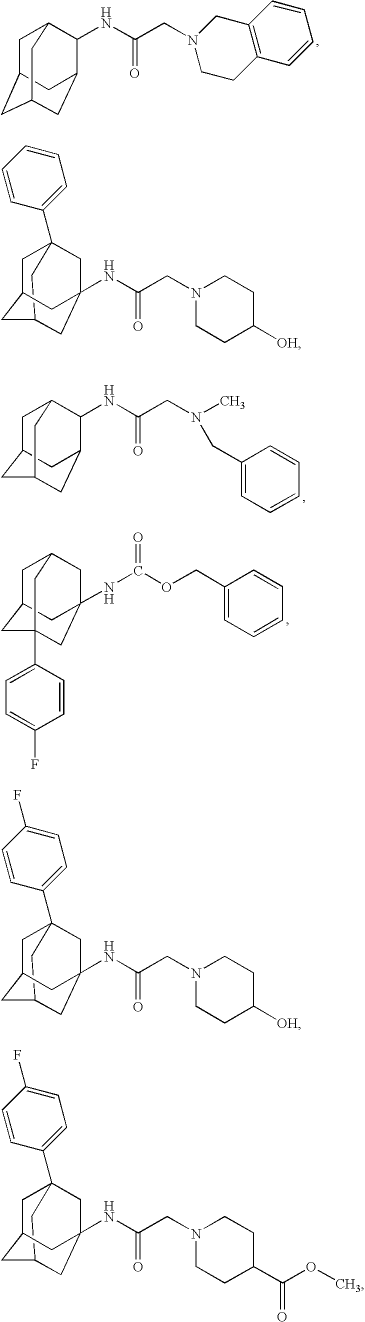 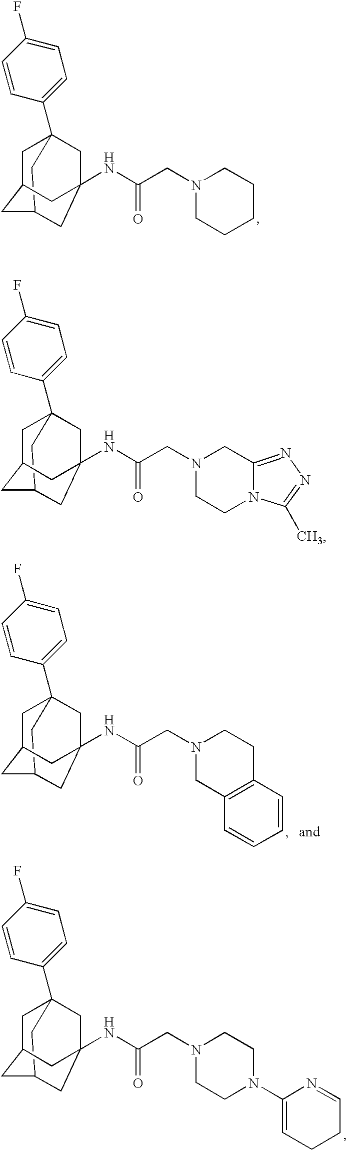
or pharmaceutically acceptable salts thereof.

7. A compound selected from the group consisting of: 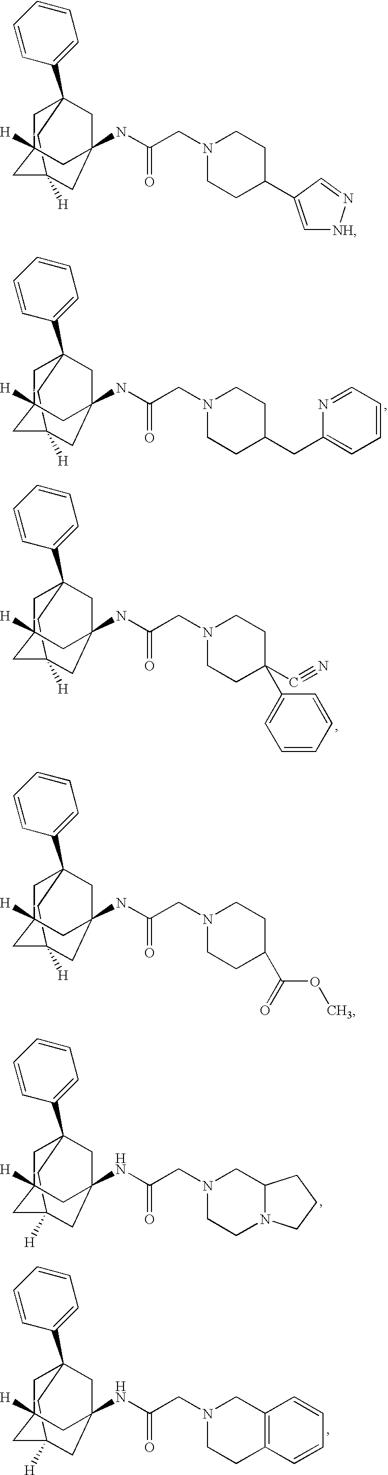 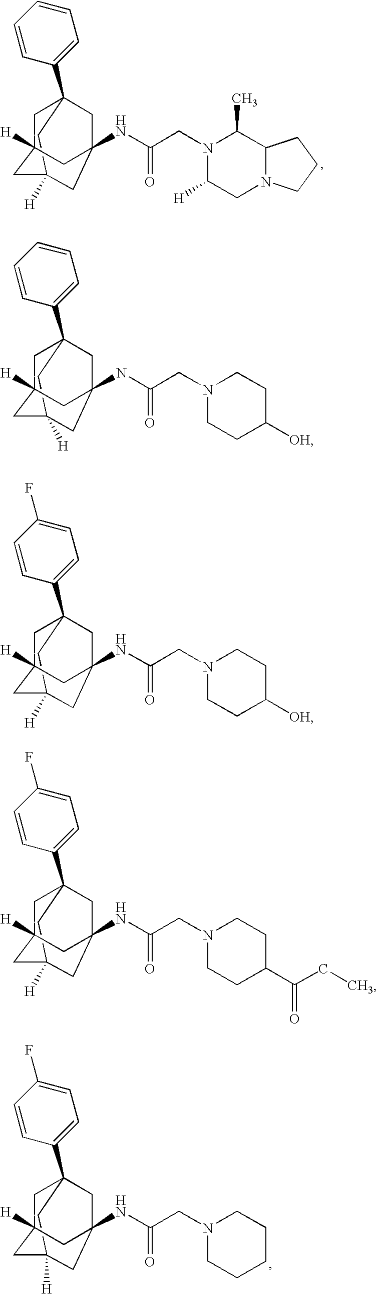 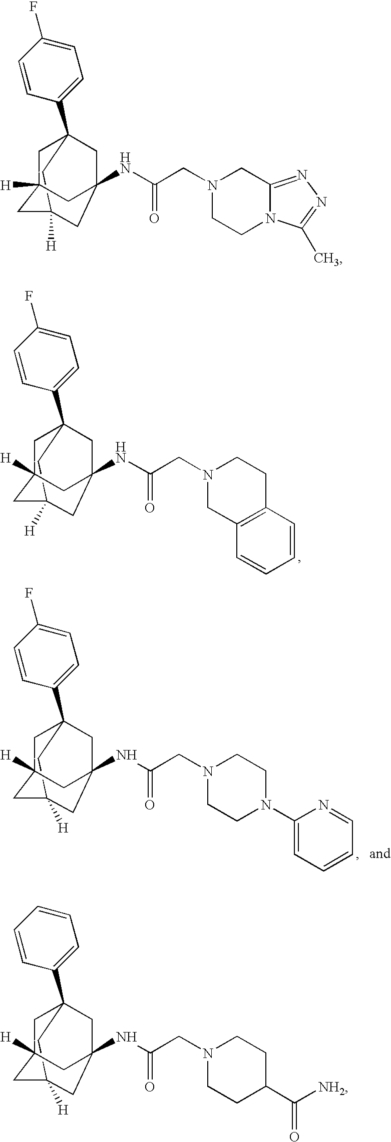
or pharmaceutically acceptable salts thereof.

8. A pharmaceutical composition comprising an effective amount of a compound according to claim 1, or a pharmaceutically acceptable salt or solvate thereof, and a pharmaceutically acceptable carrier.

9. A method of treating a condition that is mediated by the modulation of the 11-β-hsd-1 enzyme, the method comprising administering to a mammal an effective amount of a compound according to claim 1, or a pharmaceutically acceptable salt or solvate thereof.

10. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which liver is a target organ, the method comprising administering to a mammal an effective amount of a compound according to claim 1 or a pharmaceutically acceptable salt or solvate thereof.

11. A method of treating a condition that is mediated by the modulation of the 11-β-hsd-1 enzyme, the method comprising administering to a mammal an effective amount of a compound, according to claim 1, in combination further comprising a therapeutic agent to treat glaucoma, or a pharmaceutically acceptable salt or solvate thereof.

12. The method of treating a condition according to claim 11, comprising administering to a mammal an effective amount of a compound according to claim 1, in combination with a prostanoid receptor agonist, wherein said agonist is latanoprost, to treat glaucoma, or a pharmaceutically acceptable salt or solvate thereof.

13. The method of treating a condition according to claim 11, comprising administering to a mammal an effective amount of a compound according to claim 1, in combination with a known therapeutic agent, wherein said agent is a carbonic anhydrase inhibitor, to treat glaucoma or a pharmaceutically acceptable salt or solvate thereof.

14. The method of treating a condition according to claim 11, comprising administering to a mammal an effective amount of a compound according to claim 1, in combination with a known therapeutic agent, wherein said therapeutic agent is a PPAR agonist, to treat diabetes.

15. A method of preparing a compound of formula (III) 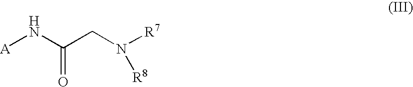
wherein:
R⁷ and R⁸ are independently selected from the group consisting of H, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl; or
R⁷ and R⁸ may optionally be taken together with the nitrogen to which they are attached to form a (4-11)-membered heterocyclic which may be fused or unfused;
t is selected from the group consisting of 0, 1, 2, 3, 4, and 5;
each R⁹ and R¹⁰ is independently selected from the group consisting of H, (C₁-C₆) alkyl, (C₂-C₆) alkenyl, and (C₂-C₆) alkynyl;
A is adamantyl, unsubstituted or substituted with 1 to 5 substituents independently selected from the group consisting of halo, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl;
comprising the steps of:
treating a compound of formula (II) 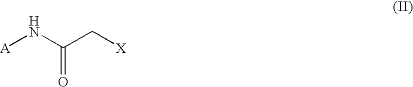
wherein:
X is a leaving group; and
A is defined as above;
with an amine in a solvent in the presence of a base to produce a compound of formula (III); and
(b) treating a compound of formula (Ia)
A—NH₂ (Ia)
wherein:
A is defined as above;
with an acyl halide in a solvent in the presence of a base to produce a compound of formula (II).

16. The method according to claim 15, wherein X in step (a) is selected from group consisting of Cl, Br, and methanesulfonate.

17. The method according to claim 15, wherein the amine in step (a), is R⁷R⁸NH.

18. The method according to claim 15, wherein the base in step (a) is selected from the group consisting of K₂CO₃, NaHCO₃, and (C₂H₅)₃N.

19. The method according to claim 15 wherein step (a) proceeds at a temperature range from about 20 degrees Celsius to the boiling point of the solvent.

20. The method according to claim 15, wherein the solvent in step (b) is CH₂Cl₂ or acetonitrile.

21. The method according to claim 15, wherein the base in step (b) is (C₂H₅)₃N or NaHCO₃.

22. The method according to claim 15, wherein step (b) proceeds at a temperature range from about −15 degrees Celsius to about 50 degrees Celsius.

23. A method of preparing a compound of formula (IIa) 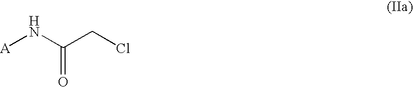
wherein:
A is adamantyl, unsubstituted or substituted with 1 to 5 substituents independently selected from the group consisting of halo, (C₁-C₆) alkyl, (C₂-C₆) alkenyl, (C₂-C₆) alkynyl, (CR⁹R¹⁰)ₜ(C₃-C₁₀)cycloalkyl, (CR⁹R¹⁰)ₜ(C₆-C₁₀)aryl, and (CR⁹R¹⁰)ₜ(4-11)-membered heterocyclyl;
each R⁹ and R¹⁰ is independently selected from the group consisting of H, (C₁-C₆) alkyl (C₂-C₆) alkenyl, and (C₂-C₆) alkynyl;
t is selected from the group consisting of 0, 1, 2, 3, 4, and 5;
comprising the steps of:
(c) treating a compound of formula (V) 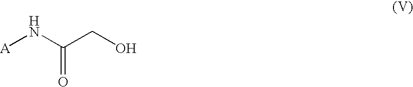
wherein:
A is defined as above;
with neat SOCl₂ or SOCl₂ in a solvent to form a compound of formula (IIa);
(d) treating a compound of formula (IV) 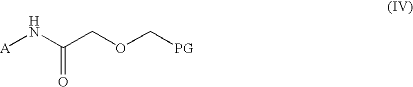
wherein:
A is defined as above;
PG is protecting group;
with a protecting group removing agent in a solvent to form a compound of formula (V);
and
(e) coupling a compound of formula (Ia) A-NH₂
A-NH₂ (Ia)
wherein:
A is defined as above;
with an acid to form a compound of formula (IV).

24. The method according to claim 23, wherein the solvent in step (c) is CCl₄.

25. The method according to claim 23, wherein step (c) is performed at a temperature from 20 degrees Celsius to 100 degrees Celsius.

26. The method according to claim 23, wherein PG in step (d) is (C₆-C₁₂) aryl.

27. The method according to claim 26, wherein the aryl is phenyl.

28. The method according to claim 23, wherein the protecting group removing agent in step (d) is (CH₃)₃Sil.

29. The method according to claim 23, wherein the solvent in step (d) is CHCl₃.

30. The method according to claim 23, wherein step (d) is performed at a temperature from 20 degrees Celsius to the boiling point of the solvent.

31. The method according to claim 23, wherein the acid in step (e) is benzyloxyacetic acid.

32. A method of preparing a compound of formula (III) 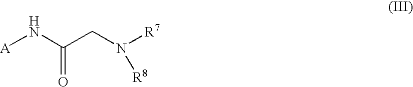
wherein:
R⁷ and R⁸ are independently selected from the group consisting of H, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl; or
R⁷ and R⁸ may optionally be taken together with the nitrogen to which they are attached to form a (4-11) membered heterocyclic which may be fused or unfused;
each R⁹ and R¹⁰ is independently selected from the group consisting of H, (C₁-C₆) alkyl, (C₂-C₆) alkenyl, and (C₂-C₆) alkynyl;
A is adamantyl, unsubstituted or substituted with 1 to 5 substituents independently selected from the group consisting of halo, (C₁-C₆) alkyl, (C₂-C₆) alkenyl, (C₂-C₆) alkynyl, —(CR⁹R¹⁰)ₜ(C₃-C₁₀)cycloalkyl, —(CR⁹R¹⁰)ₜ(C₆-C₁₀)aryl, and —(CR⁹R¹⁰)ₜ(4-11)-membered heterocyclyl;
t is selected from the group consisting of 0, 1, 2, 3, 4, and 5;
comprising the steps of:
(f) treating a compound of formula (Ia) A-NH₂ with a compound of formula (VI) 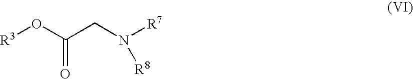
wherein:
R³ is (C₁-C₆) alkyl;
R⁷ and R⁸ are defined above;
in the presence of a reagent in a suitable solvent to form a compound of formula (III);
(g) treating a compound of formula (VII) 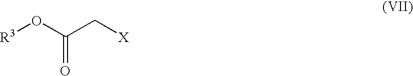
wherein:
R³ is defined above;
X is a leaving group;
with an amine in a suitable solvent in the presence of a base to form a compound of formula (I).

33. The method according to claim 32, wherein step (f) R³ is methyl or ethyl.

34. The method according to claim 32, wherein X in step (f) is selected from the group consisting of Cl, Br and methanesulfonate.

35. The method according to claim 32, wherein step (f) is performed with the reagent Al(CH₃)₂Cl.

36. The method according to claim 32, wherein step (f) is performed with the solvent CH₂Cl₂.

37. The method according to claim 32, wherein step (f) is performed at a temperature from 0 degrees Celsius to about 20 degrees Celsius.

38. The method according to claim 32, wherein the amine in step (g) is R⁷R⁸NH.

39. The method according to claim 32, wherein the solvent in step (g) is CH₂Cl₂ or DMF.

40. The method according to claim 32, wherein the base in step (g) is NaHCO₃ or triethylamine.
